(19) United States
(12) Patent Application Publication
(10) Pub. No.: US 2022/0142516 A1
(43) Pub. Date: May 12, 2022

(54) METHOD AND SYSTEMS FOR USING SURROGATE MARKERS TO IMPROVE NUTRITION, FITNESS, AND PERFORMANCE

(71) Applicant: NUEON INC., Menlo Park, CA (US)

(72) Inventors: Robert G. MESSERSCHMIDT, Menlo Park, CA (US); Hershel MACAULAY, Palo Alto, CA (US); Dara ROUHOLIMAN, San Francisco, CA (US)

(73) Assignee: NUEON INC., Menlo Park, CA (US)

(21) Appl. No.: 17/451,363

(22) Filed: Oct. 19, 2021

Related U.S. Application Data
(60) Provisional application No. 63/198,720, filed on Nov. 6, 2020.

(51) Int. Cl. A61B 5/145; A61B 5/00

(52) U.S. Cl. CPC A61B 5/14; A61B 5/14532; A61B 5/4872; A61B 5/0075

(57) ABSTRACT

A blood sample collector can be used to collect a blood sample from a subject. The blood sample collector can be placed in a receptacle of a spectrometer to measure spectral data from the blood sample while the blood sample separates. The container may comprise a window to allow light such as infrared light to pass through the container, with the blood sample at least partially separating within the container between spectral measurements, which can provide improved accuracy of the measurements and additional information regarding the sample. Measurements of the level of a biomarker surrogate in a person's blood may be used to assist the person to make beneficial changes to their diet, exercise regimen, or other aspects of their lifestyle.

### RELATED APPLICATIONS

This application claims the benefit under 35 U.S.C. § 119(e) of U.S. Provisional Patent Application No. 63/198,720, filed Nov. 6, 2020, entitled “Method and Systems for Using Surrogate Markers to Improve Nutrition, Fitness, and Performance”, the entire disclosure of which is incorporated herein by reference.

The subject matter of this patent application is related to PCT/US2019/030052, filed Apr. 30, 2019, entitled “Systems and Methods for Blood Analysis”, and PCT/US2016/026825, filed Apr. 8, 2016, entitled “METHOD AND APPARATUS FOR DETERMINING MARKERS OF HEALTH BY ANALYSIS OF BLOOD”, the entire disclosures of which are incorporated herein by reference.

### BACKGROUND

Prior approaches to health care can be less than ideal in at least some respects. Over three trillion dollars is spent annually on health care, and many of the resources spent on health care are directed to reactive treatment to preventable diseases. It would be beneficial to have improved measurements of subjects that would allow preventive measures to be taken, such as modifications in diet and lifestyle. Also, the prior paradigm of health care is based substantially around averages, yet most people are not average in at least some respects. Although some efforts have been made to personalize healthcare, the effectiveness of personalized care can be limited to the accuracy and frequency of data available for a given subject. Also, many people wish to improve their performance, appearance, or both, and such people could benefit from improved information about their wellness and physical conditioning, even though such people may not need medical care or be at risk of disease.

The prior approaches to measuring health and wellness of a subject can be less than ideal in at least some respects. Although wearable devices such as smart watches have been proposed to measure the heart rate and activity of the subject, these devices can provide somewhat limited information in at least some respects. Although laboratory-based methods such as blood panels can be ordered by medical personnel, these blood tests tend to be time consuming, rely on visits to a testing facility, can be expensive, and tend to be taken less frequently than would be ideal. Also, the amount of blood drawn with the prior approaches can limit the frequency and willingness of the subject to provide blood at more frequent intervals.

Work in relation to the present disclosure suggests that prior approaches to health and wellness may have placed more reliance on biomarkers than would be ideal. For example, biomarkers may be more difficult to detect than would be ideal in at least some instances. Also, presenting biomarker data may result in more regulatory compliance than is helpful for health and wellness applications, which may be associated decreased availability of biomarker data in at least some instances.

Work in relation to the present disclosure suggests that it would be helpful to provide more frequent blood measurements that would allow a subject to test the effect of his or her behavior on markers of health. Also, more frequent blood measurements of people could allow the identification of new markers. Further, work in relation to the present disclosure suggests that measurements of surrogates for biomarkers may be used to generate recommendations to a person to make a change in their nutrition, exercise regimen, or lifestyle in order to improve their health.

In light of the above, there is a need for improved blood testing that can be performed more frequently, with decreased amounts of blood, and with sufficient accuracy and repeatability to provide meaningful information regarding the health and wellness of the subject.

### SUMMARY

The presently disclosed methods and apparatus allow frequent, reliable blood measurements with small amounts of blood, typically less than a drop of blood, such that the blood sample can be obtained in a relatively painless manner. In some embodiments, the blood sample is obtained with a blood sample collector comprising a container with a volume within a range from about 0.2 microliter to about 5 microliters. The blood collector and a spectrometer can be configured for whole blood reagentless spectroscopy, in which the blood sample separates within the container when the collector has been placed on the receptacle of the spectrometer. The blood collector and spectrometer can be used in many applications, such as quantitative measurements of blood chemistry and wellness applications that do not rely on quantitative measurements of blood chemistry.

The blood collector and spectrometer described herein may be used to analyze a blood sample or samples to determine a level of a surrogate for a biomarker in a person's blood. In some embodiments, the surrogate is a level of fat in the blood of the person, and the level of fat and its variability serve as an indicator of the level of triglycerides in the person's blood. The correlation between measurements of the surrogate and the level of triglycerides may be used to generate a recommendation to the person regarding a change in their intake of food, their exercise regimen, or another aspect of their lifestyle.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

Better understanding of the features, advantages and principles of the present disclosure will be obtained by reference to the following detailed description that sets forth illustrative embodiments, and the accompanying drawings of which:

FIG. 1 shows a diagram of an exemplary blood sample and spectrometer, in accordance with some embodiments;

FIGS. 2A and 2B show block diagrams of an exemplary spectrometer measuring spectra of a separating blood sample, in accordance with some embodiments;

FIG. 3 shows a diagram of an exemplary blood sample collector, in accordance with some embodiments;

FIG. 4 shows a block diagram of an exemplary spectrometer with network connectivity, in accordance with some embodiments;

FIG. 5 shows exemplary wavelength plots over time from a spectrometer, in accordance with some embodiments;

FIG. 6 shows a flowchart of an exemplary blood spectroscopy process, in accordance with some embodiments;

FIG. 7 shows a block diagram of an exemplary system comprising a blood spectrometer with network connectivity, in accordance with some embodiments;

FIGS. 8 to 14B show exemplary graphical user interfaces, in accordance with some embodiments;

FIGS. 15A to 16B show exemplary messages displayed to a user via a graphical user interface, in accordance with some embodiments;

FIG. 17 shows an exemplary progress chart of a user displayed via a graphical user interface, in accordance with some embodiments;

FIG. 18 shows an exemplary social networking platform, in accordance with some embodiments;

FIG. 19 shows a flowchart of an exemplary process, in accordance with some embodiments;

FIG. 20 shows a method of spectral data analysis suitable for incorporation with some of embodiments; and

FIG. 21 shows the final selection of wavelengths of a red blood cell spectrum, optimized using a genetic algorithm procedure, in accordance with some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The presently disclosed methods and apparatus will find application in many fields. Although reference is made to testing blood, the presently disclosed methods and apparatus can be used to test many types of biomatrices. The measured biomatrix may comprise a bodily fluid, such as urine, saliva, tears (lacrimal fluid), interstitial fluid, or sweat, for example. The presently disclosed methods and apparatus can also be used to measure other materials and biomatrices, such as sebum and fecal material. Work in relation to the present disclosure suggests that fat, the microbiome, and other material present in fecal samples can be related to dietary health such as sufficiency or overload, and the methods and apparatus disclosed herein are well suited to measuring amounts of fat in a fecal biomatrix.

The presently disclosed methods and apparatus can be incorporated into prior methods and apparatus. For example, although reference is made to a scanning digital mirror, the presently disclosed methods and apparatus can be combined with other types of spectroscopy such as Fourier Transform Infrared (FTIR) spectroscopy, and dispersive spectrometers. For example, the blood collector as disclosed herein can be combined with one or more components of FTIR spectroscopy or dispersive spectroscopy, and combinations thereof. By way of example, the presently disclosed spectrometer may comprise one or more components of the commercially available DLP NIRSCAN Evaluation Module, commercially available from Texas Instruments.

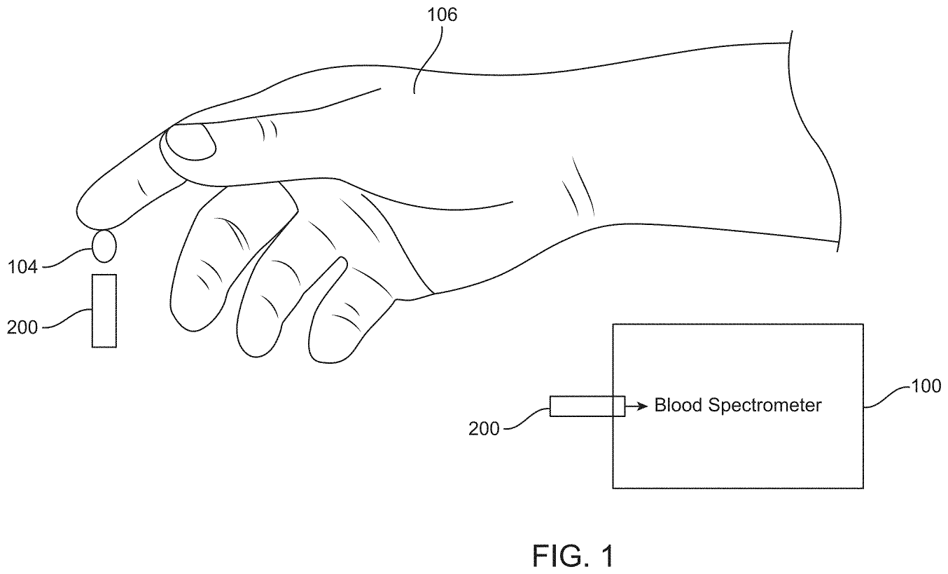

Turning now to FIG. 1, a diagram of an exemplary blood sample 104 and a spectrometer 100 is shown, in accordance with some embodiments. In this embodiment, the spectrometer 100 is configured to receive the sample of blood 104 from a finger of the user 106, although the blood may be sampled from other locations, such as the forearm of the user. In some embodiments, the sample of blood 104 is typically less than a drop of blood which has a volume of about 50 microliter (μL). The sample may comprise a volume within a range from about 0.2 μL to about 5 and the amount of blood can be within a narrower range from about 0.5 μL to about 2 μL, e.g. about 1 μL. The small amount of blood allows the blood sample to be taken from locations that are less painful for the subject. Once a sample of blood 104 is taken from the user 106, the sample holder 200 may be inserted into the spectrometer 100 for spectral analysis. Other bodily fluids can be as described herein sampled similarly with modification of the sampling device. Other materials as described herein can be sampled and measured by the user.

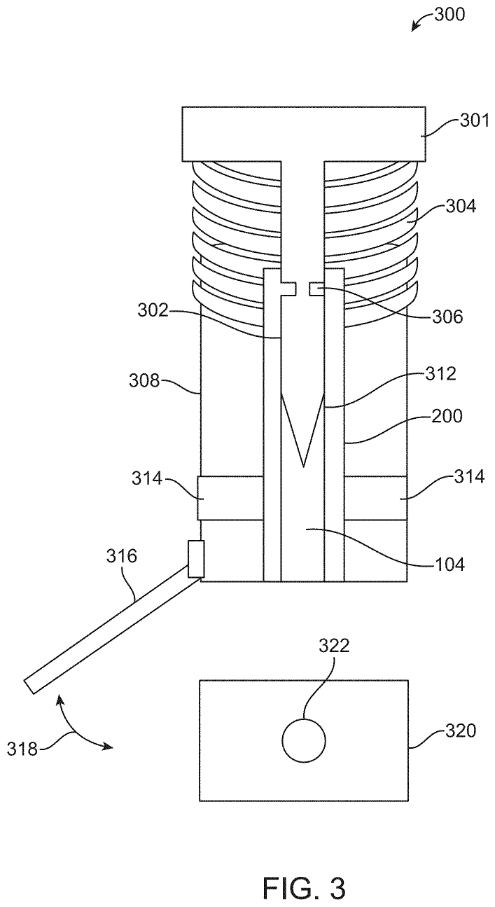

The amount of innervation of the skin can vary depending on the location, and the blood can be drawn at a location of the subject with decreased innervation. For example, the sample can be drawn from the user's forearm 106 with a sample holder, an example of such is shown and described below in FIG. 3. The sample holder may be placed in a receptacle of the spectrometer 100. The sample holder may be configured to allow the blood 104 to at least partially separate into various components. As the blood 104 separates or at least partially separates within the sample holder, the spectrometer 100 may selectively measure the various components of the blood 104, and generate a plurality of wavelength spectrum plots and/or other spectral data that correspond to the separation of the components of the blood 104. For example, the spectrometer 100 and/or some other processing system may generate spectral data of the sample at a plurality of wavelengths and a plurality of times corresponding to at least a partial separation of the sample of blood 104 into a plurality of components of the sample.

The sample of blood can be combined with an anti-coagulant or blood thinner to decrease clotting when the blood sample has been placed in the sample holder. This can allow the sample to settle gravimetrically without substantial clotting. For example, the sample holder may comprise an anticoagulant prior to placing the blood in the sample holder. The anticoagulant may comprise one more commercially available anticoagulants, such as heparin. In embodiments where it is desirable to measure a clotting rate, the sample can be measured without or with reduced amounts of anticoagulant in order to allow at least partial clotting of the blood.

The at least partial separation of the blood in the sample holder may occur gravimetrically in response to the earth's gravitational field, and in some embodiments without spinning the sample in a centrifuge. The separation can be related to differences in density of components of the blood. The red blood cells, which contain iron, tend to settle toward the bottom of the sample holder, and the blood plasma, which is less dense than the red blood cells, tends to form near an upper portion of the container. In some embodiments, white blood cells settle in a region between the red blood cells and plasma. The spectrometer and processor can be configured to measure this region. The white blood cells and other cells that contain the cellular DNA, tend to settle in a region between the plasma and the settled red blood cells. This region can be referred to as the “buffy coat.” Infrared spectra from this region of the tube comprising the separated blood can provide information related to DNA changes such as methylation. The spectrometer can be configured to measure at least 3 regions of the blood sample, a first region corresponding to the plasma, a second region corresponding to white blood cells and a third region corresponding to red blood cells. The spectrometer can be configured to selectively scan each of these three regions with a plurality of successive measurements at appropriate times as described herein.

To illustrate, the bodily fluid as described herein such as blood may be drawn into the sample holder via a capillary action. As the bodily fluid such as blood is stored in the sample holder, the components of the bodily fluid such as blood 104 may separate into a plurality of components, for example, plasma, red blood cells, white blood cells, etc. The spectrometer 100 may illuminate each of the components by directing light to appropriate locations in the illumination window. With the gravimetric separation as described herein, the ratios of the components of blood at different locations in the sample may change, even though the blood sample may not fully separate. In response to the at least partial separation, each illuminated region of the sample may yield a different wavelength spectrum (e.g., multiple wavelengths of light with varying intensities). The spectrometer 100 may detect these various wavelength spectra (e.g., via an optical detector) and the spectrometer 100 or other elements of the system may process the spectra for analysis (e.g., determine a relative change in a surrogate of a biomarker over time, generate a graphical display of the wavelength spectrums over time, provide medical advice to a person who provided the blood sample, provide general healthcare advice, etc.). In this regard, the spectrometer 100 or a system of which it is a part may include a processor and various forms of associated hardware, software, firmware, and combinations thereof.

The spectrometer 100 may focus light on a particular region within the illumination window of the sample holder and produce various wavelength spectra. Alternatively, or in combination, a portion of the sample may be imaged onto a detector through the window of the blood collector. For example, the sample holder may separate the blood 104 into its various components. The spectrometer 100 may illuminate the blood 104 and each of its components, at a particular location within the illumination window, as the blood 104 and its components separate within the sample holder. The detector and the processor of the spectrometer 100 may then generate various wavelength spectra over time, which can be processed accordingly.

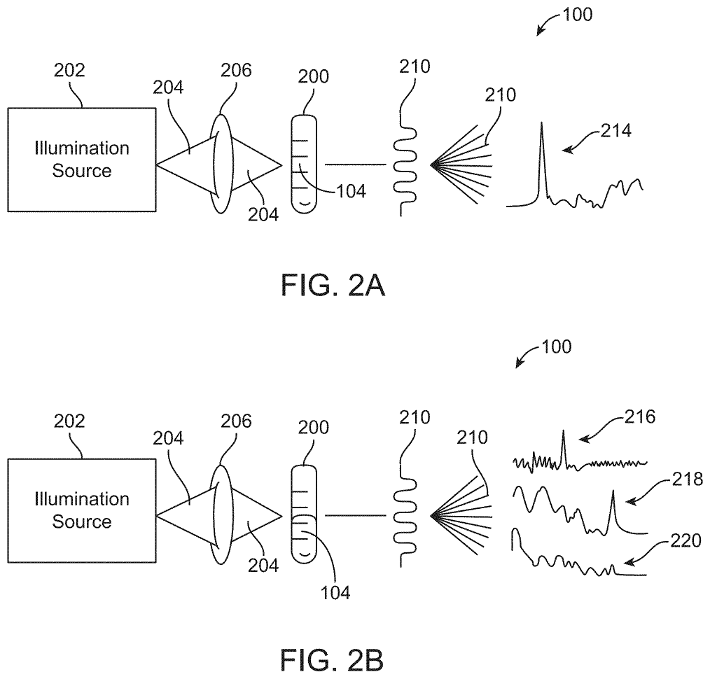

FIGS. 2A and 2B show block diagrams of the exemplary spectrometer 100 measuring spectra (e.g., spectra 214, 216, 218, and 220) of a separating blood sample 104, in accordance with some embodiments. More specifically, FIG. 2A illustrates the spectrometer 100 illuminating the sample blood 104 in a manner that may provide an initial assessment of the blood 104 over a shorter period of time (e.g., within one minute of being drawn). An illumination source 202 of the spectrometer 100 may propagate light 204 through an optical configuration 206 (e.g., various lenses, diffraction grating, mirrors, etc.). The optical configuration 206 may then selectively focus the light 204 to one or more locations of an illumination window of the sample holder 200, which, in turn, produces various wavelengths 210 of light 204. These wavelengths may be detected by an optical detector configured with the spectrometer 100 so as to produce a wavelength spectrum 214.

FIG. 2B shows a similar embodiment where blood 104 has at least partially separated into its various components within the sample holder 200 after a longer period of time (e.g. after at least about 5 minutes of being drawn from the subject). The sample such as a blood sample can be drawn into an elongate transparent structure such as a glass tube, for example. The sample holder 200 may remain in a receptacle of the spectrometer while the blood 104 separates within the holder.

A processor may direct the optical configuration 206 to focus the light 204 at various locations of the illumination window of the sample holder 200 as the blood 104 separates within the sample holder 200. For example, a first ratio of components of the blood may exist at a first location within the sample holder 200, a second ratio of components of the blood 104 may exist at a second location within the sample holder 200, and so on, as the blood separates within the sample holder 200.

The processor may direct the optical configuration 206 to focus the light 204 at the various locations to produce different wavelength spectra.

If the blood sample is left undisturbed in the sample holder for a sufficient amount of time, the blood sample may separate into layers corresponding to specific components of the blood sample. In this example, the spectrometer 100 produces an upper wavelength spectrum 216 representative of a user's plasma within the blood 104 at an upper location of the sample holder 200, a wavelength spectrum 218 representative of the user's white blood cells within the blood 104 at an intermediate location within the sample holder 200, and a wavelength spectrum 220 representative of the user's red blood cells within the blood 104 at a lower location within sample holder 200. Although the blood 104 is shown fully separated into different components, work in relation to embodiments of the present disclosure suggests that partial separation of blood is sufficient to provide useful information. Thus, the spectrometer 100 may provide a more in-depth analysis of the user's blood 104.

Again, the illumination source 202 and the optical configuration 206 may alternatively or additionally focus light 204 to a particular location on the illumination window of the sample holder 200. In this embodiment, the sample holder 200 may separate the blood 104 into its various components and propagate those components through the sample holder 200 over time. Thus, the spectrometer 100 may in essence take “snapshots” of the blood 104 and its components over time to generate the wavelength spectra 216, 218, and 220.

One advantage of the spectrometer 100 exists in the placement of the optical configuration 206 between the illumination source 202 and the sample holder 200. For example, by placing the optical configuration 206 between the illumination source 202 and the sample holder 200, the spectrometer 100 may decrease heating of the blood 104 within the sample holder 200. That is, the spectrometer 100 may distance the sample holder from the illumination source 202 in such a way that the blood 104 within the sample holder 200 does not overheat, which can result in measurement errors. In some embodiments, the spectrometer is configured to heat the blood sample by no more than about 5 degrees centigrade when the sample has been placed in the spectrometer and measured for an extended period of time, e.g., for 5 minutes. This limit to heating of no more than 5 degrees C. when placed in the spectrometer for 5 minutes while the blood separates can be helpful for whole blood reagentless measurements.

Although reference is made to scanning the measured region of the blood sample in FIG. 2B, in some embodiments, the sampled region of blood remains fixed while the blood separates. For example, the light beam can be focused to a location of the sample holder 200 such as an upper location, or collimated light may be passed through a window of the sample holder. The spectra can be recorded as the blood separates at least partially. In some embodiments, the measurement location comprises an upper location of the blood sample. As the blood separates, the red blood cells move away from the upper portion of the column of blood, and the spectra of the upper portion becomes more consistent with spectra of the blood plasma. In some embodiments, measurements of the blood sample from a single location can provide spectral information from the whole blood of the sample and the plasma. Also, the plasma and whole blood information can be used to determine spectral properties of the lower portion of the sample related to a hematocrit of the blood sample based on changes to the spectral signal at the upper location of the blood sample, for example based on subtraction of spectral signals.

Alternatively, or in combination, the measured portion of the blood sample may remain fixed at a lower portion of the blood sample below a midpoint of the blood column. As the blood separates, additional red blood cells are located at the lower portion of the blood column and the sample becomes more consistent with spectra of a hematocrit.

The blood sample can remain placed in the spectrometer for an appropriate amount of time for at least partial separation of the blood to occur, for example gravimetrically. The container may comprise a sealed container to decrease, or even inhibit, drying of the sample (such as a blood sample) during the gravimetric separation. The sample can be allowed to separate for an appropriate amount of time, which can be as short as five minutes, although the separation time may be longer. For example, the amount of time can be within a range from about 5 minutes to about 3 hours, and more specifically from about 30 minutes to 2.5 hours, and for example within a range from about 1 hour to about 2 hours.

The processor can be programmed with instructions for other ranges. For example, the processor can be configured with instructions to spectroscopically measure the sample at a plurality of times within a range from about 5 minutes to about 3 hours while the sample separates, and optionally within a range from about 20 minutes to about 2 hours, and further optionally within a range from about 30 minutes to about 1.5 hours.

A plurality of measurements can be obtained during the time the sample is allowed to separate gravimetrically. The plurality of measurements may comprise successive measurements obtained with an interval of approximately 30 seconds to 10 minutes between measurements, and for example, 1 minute to 5 minutes between successive measurements.

In some embodiments the detector comprises a plurality of detectors as described herein, in which each detector corresponds to a location of the blood sample. For example, a pair of detectors can be used to measure the blood sample at a pair of fixed locations as the blood sample separates, e.g., at an upper location and at a lower location of the blood sample. A grating, digital mirror, interferometer, or other wavelength selector may be scanned or operated to determine the spectra of the sample at the pair of locations.

The spectrometer can be configured in many ways, and may comprise one or more components of known spectrometers, such as a Fourier Transform Infrared (FTIR) spectrometer, a dispersive spectrometer with a detector array, or a spectrometer with a tunable laser as described in U.S. application Ser. No. 14/992,945, filed on Jan. 11, 2016, entitled “Spectroscopic measurements with parallel array detector”, published as US20160123869A1 on May 5, 2016, the entire disclosure of which is incorporated herein by reference. In some embodiments the spectrometer comprises a tunable laser, for example.

FIG. 3 shows a block diagram of an exemplary blood sample collector 300, in accordance with some embodiments. The blood sample collector 300 comprises a housing 308 to support structures of the blood collector 300. The blood sample collector 300 may comprise a lancet needle 302. The lancet needle 302 may be made from many materials, including but not limited to surgical grade steel. The lancet needle 302 may be sized to extend out of an end of a tube 312 configured within the sample holder 200. The sample holder 200 may be configured within housing 308 of the blood sample collector 300. A user may thus depress the lancet needle 302 through the opening of the sample holder 200 to draw the blood 104 from the user (or another) by pushing a button 301 affixed to an end of the lancet needle 302. In this regard, the blood sample collector 300 may also include a spring mechanism 304 that allows a user to depress the lancet needle 302 into the user's skin to penetrate the user's skin. When the user releases pressure from the button 301, the spring 304 retracts the lancet needle 302 from the user's skin thereby drawing blood 104 into the tube 312 of the sample holder 200 (e.g., via capillary action, suction, or the like).

In some embodiments, the tube 312 comprises a substantially transparent elongate container comprising an elongate axis to separate the sample of blood into the plurality of components. In some embodiments, the tube 312 comprises a capillary tube configured to separate the sample of blood into the plurality of components. In some embodiments, the tube 312 has a volume within a range from about 0.5 to about 2.0 microliter

The amount of retraction may be limited by an O-ring groove 306 in which an O-ring may be disposed. For example, when the user releases pressure from the button 301 and the lancet needle 302 retracts, the O-ring may limit the amount of retraction to the upper portion of the sample holder 200, thereby retaining the lancet needle 302 within the blood sample collector 300.

When the blood 104 is retained within the sample holder 200 of the blood sample collector 300, the blood sample collector 300 may be closed and/or otherwise sealed with a lid 316. For example, the lid 316 may be attached to the blood sample collector 300 via a hinge mechanism that allows the blood sample collector 300 to open and close as indicated by the angular direction 318. The lid 316 may close the blood sample collector 300 via a compression fit, or other attachment mechanism. However, other embodiments may include attaching the lid 316 to the blood sample collector without a hinge (e.g., via compression fit or other attachment mechanism).

Also illustrated in this embodiment is a guide mechanism 320 that may allow the blood sample collector 300 to accurately draw the blood of the user 104 from a specified location on the user's skin. For example, the guide mechanism 320 may comprise an adhesive that sticks to the user's skin. The guide mechanism 320 may comprise an aperture 322 that is approximately the same size as the tube 312 through which the lancet needle 302 traverses. Thus, when the blood sample collector 300, and more specifically the tube 312, is placed proximate to the user's skin in the aperture 322 of the guide mechanism 320, the lancet needle 302 may penetrate the user's skin through the aperture 322.

The tube 312 may be configured of an optically transparent material, such as glass, plastic or the like. The blood sample collector 300 may be configured with optical ports 314 such that light from an illumination source, such as the illumination source 202 of FIG. 2A or 2B, may pass through the blood sample collector 300 and through the blood 104 to a detector of the spectrometer 100 for subsequent wavelength spectra processing. In this regard, the sample holder 200 may also have optical ports 314 and/or be configured from an optically transparent material capable of propagating light through the blood 104 contained within the tube 312. In some embodiments, the sample holder 200 comprises a slit aperture configured to direct light through the substantially transparent tube 312. The long axis of the slit aperture may be aligned with a long axis of transparent tube 312.

In some embodiments, the sample holder 200 may be configured to provide reagentless whole blood spectroscopy. In some embodiments, a volume of the sample holder 200 is within a range from about 0.25 microliters to about 4 microliters and optionally within a range from about 0.5 to about 2 microliters. In some embodiments, a height of a window in the sample holder 200 is within a range from about 1 mm to about 20 mm, and optionally within a range from about 2 mm to about 10 mm.

The sample holder 200 may be placed in the spectrometer with or without sample collector 300. In some embodiments, sample collector 300 is placed in the spectrometer with the lancet 302 within the sample holder 200 and the spectra measured. The spectrometer may comprise a receptacle sized and shaped to receive the sample collector 300. The receptacle of the spectrometer may comprise a channel sized and shaped to receive the housing 308 of the sample collector 300. Alternatively, or in combination, the receptacle of the spectrometer may be sized and shaped to receive the sample holder 200 without the housing of the sample collector.

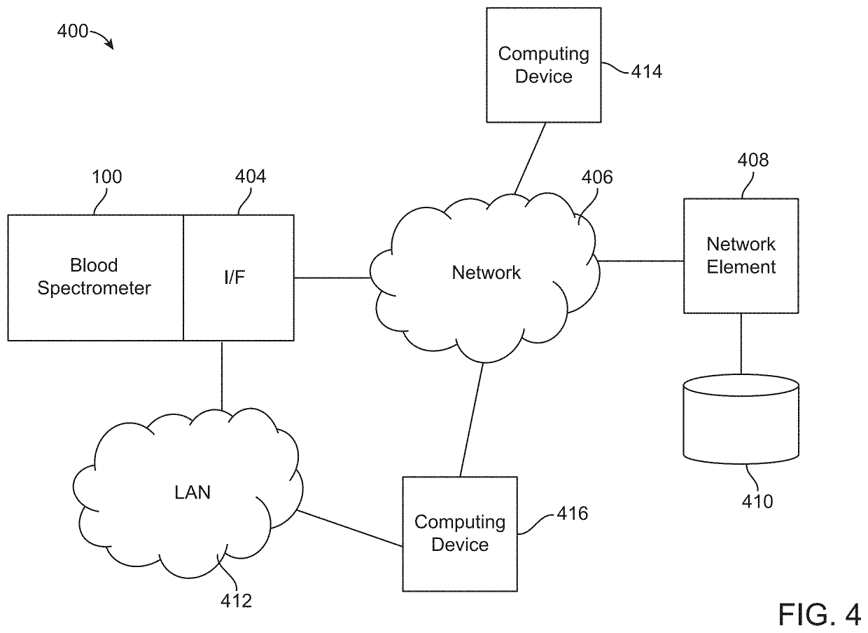

FIG. 4 shows a block diagram of an exemplary spectrometer 100 with network connectivity, in accordance with some embodiments. The spectrometer 100 may be configured with, or coupled to, a network interface 404 that is communicatively coupled to a network 406 (e.g., the Internet) and/or a local area network (LAN) 412. For example, the spectrometer 100 may be configured to receive a sample of blood contained within a sample holder, such as the sample holder 200. The spectrometer 100 may illuminate the sample of blood as the blood at least partially separates within the sample holder. A processor operatively coupled to the spectrometer 100 and/or configured with the spectrometer 100 may be configured with instructions to generate spectral data of the sample at a plurality of wavelengths and a plurality of times corresponding to at least partial separation of the sample of blood into a plurality of components of the sample.

When the spectrometer 100 detects the wavelength spectra of one or more of the components of the blood 104, the spectrometer 100 may communicate the information pertaining to the wavelength spectra and/or the spectral data (e.g., spatially resolved spectral data acquired at a plurality of times) to the network 406, which may in turn communicate the wavelength spectra and/or other spectral data to a network element 408 for subsequent processing. In this regard, the network element 408 may include, or be communicatively coupled to, a database 410 which may comprise various statistics and data pertaining to blood components that can be compared to and/or analyzed against the wavelength spectra of the blood 104. Alternatively, or additionally, the spectrometer 100 may include processing capability that communicates other relevant information pertaining to the blood 104 through the network 406 to the network element 408.

Also illustrated in this embodiment, is a computing device 414 that is communicatively coupled to the network 406. The computing device 414 may be used to perform analysis of the wavelength spectra and/or other spectral data of the blood 104 obtained from the spectrometer 100. In this regard, the computing device 414 may be in communication with the network element 408 to retrieve information pertaining to blood analysis such that a user of the computing device 414 (e.g., a medical professional, a trainer, or the like) can analyze the wavelength spectra from the spectrometer 100 and provide a diagnosis and/or other relevant information pertaining to the user's blood 104 to a user of the spectrometer 100. Examples of the computing device 414 include computers, smart phones, and the like, comprising various hardware, software, and/or firmware components for processing the wavelength spectra from the spectrometer 100.

In some embodiments where the spectrometer 100 is communicatively coupled to the LAN 412, the spectrometer 100 may be able to communicate wavelength spectra to a computing device 416. For example, the computing device 416 may also include computers, smart phones, and the like, comprising various hardware, software, and/or firmware components for processing the wavelength spectra and/or other spectral data from the spectrometer 100. In this regard, the computing device 416 may be that of the user using the spectrometer 100. For example, a user may draw his or her own blood 104 using the blood sample collector 300 of FIG. 3. The user may then input the sample of blood 104 into the spectrometer 100 to detect the various wavelength spectra of the components of the blood 104. The spectrometer 100 may then communicate the wavelength spectra to the user's computing device 416 such that the user may process the information and assess the user's own health.

The spectrometer 100, the computing devices 414 and 416, and the network element 408, either alone or in combination, may be configured with instructions (e.g., software components) that direct a processor to perform one or more types of data analyses. For example, a processor configured with the spectrometer 100, the computing devices 414 and 416, and/or the network element 408 may measure two or more of a surrogate for a biomarker (such as the fat level in the blood as described herein), a high density lipoprotein, a total cholesterol, a triglyceride or a glucose of the sample with a cross-validated standard errors of prediction (“CVSEP”) of no more than 12 mg/dL, 20 mg/dL, 40 mg/dL, 20 mg/dL, respectively, for each of the two of more of the high density lipoprotein, the total cholesterol, the triglyceride or the glucose of the sample, with the spectral data from the plurality of times corresponding to the at least partial separation of the sample. In some embodiments, the two or more measured quantities comprises three or more of the total cholesterol, the triglyceride or the glucose of the sample with the cross-validated standard errors of prediction of no more than 12 mg/dL, 20 mg/dL, 40 mg/dL, 20 mg/dL. In some embodiments, the three or more measured quantities comprises four or more of the total cholesterol components. In some embodiments, the processor is configured with instructions to determine a surrogate marker channel corresponding to one or more of the preceding biomarkers and to determine a change in the surrogate marker in response to changes in the surrogate marker channel.

In some embodiments, the processor is configured with instructions to measure the sample at a plurality of times as described herein. The processor can be configured to measure the blood sample at a plurality of times within a range from about one minute to about 3 hours (or longer) while the sample separates. The amount of time the sample is allowed to separate with measurements being obtained can be within a range from about 5 minutes to about 3 hours, more specifically from about 30 minutes to 2.5 hours, and for example, within a range from about 1 hour to about 2 hours. The plurality of measurements may comprise successive measurements obtained with an interval of approximately 30 seconds to 10 minutes between measurements, and for example, 1 minute to 5 minutes between successive measurements.

The plurality of measurements obtained with gravimetric separation can be used to measure many biomarkers with improved accuracy. It can also be used to measure one or more surrogates for a biomarker, where a surrogate refers to a measurable quantity that is strongly enough correlated to a biomarker. For example, as will be described, a level of fat in the blood plasma has been found to provide a surrogate for the triglyceride level in a person's blood. The gravimetric separation of blood can also be used to obtain the blood pressure of the patient, for example.

In some embodiments, the processor may measure one or more of a surrogate for a biomarker (e.g., a fat level in the blood, a sedimentation rate, etc.), a hormone (e.g., one or more of dehydroepiandrosterone (“DHEA”), Testosterone, Growth Hormone, Parathyroid Hormone, Estradiol, Progesterone, or Cortisol), a health and performance marker (e.g., one or more of Vitamin B12, PSA, Thyrogobulin, Troponin, IGF-1, Aldosterone, Prolactin, Creatine Kinase, Ferritin, Selenium, Homocystine, Copper, Ammonia, Folic Acid, AGE, or Cortisol), a metabolic marker (e.g., one or of Glucose, HbA1c, Glycated Albumin, Ketones, β-Hydroxybutyrate, Albumin, Total protein, BUN, Uric acid, Glutamate, GSH, Lactic Acid, CO2, pH, or Hydration), an immunology, inflammation and hematology marker (e.g., one or more of Fibrinogen, high sensitivity c-reactive protein (hsCRP), Globulins, Hematocrit, Hemoglobin, Erythrocyte sedimentation rate, Glutathione, Uric acid, Serum Amyloid A, Haptoglobin, WBC Count estimate, Transferrin saturation, Pyruvate, RBC count estimate, Platelet count estimate, Prothrombin time/INR, Interleukin-6), a cardiovascular marker (e.g., one or more of Cardiovascular total Cholesterol, HDL, LDL, Triglycerides, BNP, Apolipoprotein, or Average Blood Pressure), and a marker of stress and toxins (e.g., one or more of Creatinine, Albumin, Carboxyhemoglobin, Ethanol, Carbon monoxide, Salicylates, Acetaminophen, or Caffeine). In some embodiments, the processor is configured with instructions to determine a surrogate marker channel corresponding to one or more of the preceding biomarkers. In some embodiments, the processor is configured with instructions to determine a change in the surrogate marker in response to changes in the surrogate marker channel.

The processor can be configured with instructions to measure an aspect of the metabolism of the user or other subject from which the sample has been obtained. For example, the body's metabolism can describe the manner in which one's body processes the food that has been eaten. For example, a user may have a “slow” metabolism, and is therefore looking for ways to speed it up. Each person is different, and the methods and apparatus disclosed herein can help a user or other subject understand how their body responds to specific foods and lifestyle changes. Specific surrogates and markers of metabolism that can be measured with the methods and apparatus disclosed herein include one or more of the following: blood fat level, sedimentation rate, glucose, HbA1c (Glycated Hemoglobin), glycated albumin, ketones, O-hydroxybutyrate, albumin, total protein, blood urea nitrogen (BUN), uric acid, creatinine, glutamate, lactic acid (lactate), CO2 (bicarbonate), pH, sodium, magnesium, potassium, calcium, hydration, total body water (TBW), hematocrit, vitamin E, vitamin C, or vitamin A.

The processor can be configured with instructions to measure surrogates and markers of cardiovascular health of the user or other subject from which the sample has been obtained. Cardiovascular markers are generally related to the heart and blood vessels. Circulating biomarkers related to cardiovascular health can be identified and used to adjust lifestyle accordingly. The methods and apparatus disclosed herein can be used to measure one or more of the following surrogates or markers of cardiovascular health: blood fat level, high density lipoprotein (HDL), low density lipoprotein (LDL), total cholesterol and other cholesterol ratios, apolipoprotein, triglycerides, or average blood pressure.

The processor can be configured with instructions to measure inflammation of the user or other subject from which the sample has been obtained. Inflammation is a process by which the body protects itself from infection with foreign organisms, such as bacteria and viruses. But sometimes inflammation can become overactive and chronic, and in some instances detrimental to the health of the user. Work in relation to the present disclosure suggests that specific foods can be identified as being inflammatory. For example, sugar and other carbohydrates can be related to inflammation. The methods and apparatus as disclosed herein can be configured to allow a user to conduct an experiment related to inflammation. The processor can be configured with instructions to measure a surrogate for one or more markers of inflammation and immune function including but not limited to: fibrinogen, C-reactive protein (CRP), uric acid, serum amyloid a (0.6 mg/dl is normal but in chronic inflammation can be 10×), globulins, IgG, IgA, IgM (IgG is normally around 1000 mg/dl, but higher in food sensitivity and in multiple myeloma, and lower in immune deficiencies), or haptoglobin. In some embodiments, the processor is configured with instructions to determine a surrogate marker channel corresponding to one or more of these biomarkers and to determine a change in the surrogate marker in response to changes in the surrogate marker channel.

The processor can be configured with instructions to measure hematology of the user or other subject from which the sample has been obtained. Hematology is a measurement of the properties of blood. The hematology measurements can be indicative of dietary deficiencies. The processor can be configured with instructions to measure one or more surrogate markers of hematologic function, including but not limited to: hematocrit, hemoglobin, erythrocyte sedimentation rate (ESR), transferrin saturation (iron deficiency), pyruvate, red blood cell (“RBC”) count, white blood cell (“WBC”) count, platelet count, or prothrombin time (also referred to as INR as a measure of time for blood to clot). In some embodiments, the processor is configured with instructions to determine a surrogate marker channel corresponding to one or more of the preceding biomarkers and to determine a change in the surrogate marker in response to changes in the surrogate marker channel.

The processor can be configured with instructions to measure markers of toxins of the blood sample from the user. The toxins may comprise external factors that can negatively affect health. The toxins may comprise environmental pollutants, specific drugs, exposure to cigarette smoke, or use of alcohol for example. The processor can be configured with instructions to measure one or more markers or surrogate markers of toxins including but not limited to: carbon monoxide, carboxyhemoglobin (second hand smoke), ethanol, salicylates, acetaminophen, ethylene glycol, or caffeine.

The processor can be configured with instructions to measure markers of stress from the blood sample from the user. Stress can be reflected in blood markers and it can be helpful to decrease stress to maintain health. Insufficient sleep can be a contributing factor for stress. The processor can be configured with instructions to measure one or more markers of stress, including but not limited to: dehydroepiandrosterone (DHEA), dehydroepiandrosterone-S (DHEA-S), creatinine, glucose, C-reactive protein (CRP), fibrinogen, HbA1c, albumin, or ethanol. In some embodiments, the processor is configured with instructions to determine a surrogate marker channel corresponding to one or more of these biomarkers and to determine a change in the surrogate marker in response to changes in the surrogate marker channel.

In some embodiments, the processor is configured with instructions to measure a fecal fat channel. The fecal fat channel may comprise a channel measuring fecal fat of the user, for example with a spectrometer as disclosed herein.

The processor can be configured with instructions to enable a user to conduct an experiment with a plurality of blood samples from the user (or another subject). For example, the processor can be configured for the user to select one or more experiments as described herein, such as one or more of a metabolism experiment, a cardiovascular health experiment, an inflammation and immune function experiment, hematologic function experiment, a toxin experiment, a stress experiment, a saliva experiment, or a fecal fat experiment.

In response to the user selecting an experiment, the processor provides appropriate prompts for the user to conduct the experiment. The processor may comprise instructions to present an appropriate instruction to the user to conduct the experiment. For each type of experiment the processor can be configured with instructions to measure one or more of the surrogates or markers as described herein. The following examples of experiments list surrogates and/or markers that can be measured for each experiment, in accordance with some embodiments.

For the cardiovascular experiment, the processor can be configured with instructions to detect a change or lack of change in one or more of the following channels or corresponding surrogate channels: blood fat level, Total Cholesterol (TC), HDL, LDL, Triglycerides, very low density lipoprotein (VLDL), non-HDL, lipid ratio, B-type natriuretic peptide (BNP), apolipoprotein, or average blood pressure.

For the inflammation experiment, the processor can be configured with instructions to detect a change or lack of change in one or more of the following channels or corresponding surrogate channels: fibrinogen, ESR, hsCRP, or Globulins.

For the metabolism experiment, the processor can be configured with instructions to detect a change or lack of change in one or more of the following channels or corresponding surrogate channels: blood fat level, glucose, fructosamine, hemoglobin A1c, ketones, hemoglobin, hematocrit, insulin resistance, total protein, or albumin.

For the stress experiment, the processor can be configured with instructions to detect a change or lack of change in one or more of the following channels or corresponding surrogate channels: blood fat level, oxidized LDL (oxLDL), glutathione peroxidase, carboxyhemoblobin, carbon monoxide, creatinine, albumin, or ethanol.

For the toxin experiment, the processor can be configured with instructions to detect a detect a change or lack of change in one or more of the following channels or corresponding surrogate channels: dehydroepiandrosterone (DHEA), dehydroepiandrosterone-S (DHEA-S), creatinine, glucose, C-reactive protein (CRP), fibrinogen, HbA1c, albumin, or ethanol.

For the saliva experiment and the processor can be configured with instructions to detect a change or lack of change in one or more of the cortisol channel, or another biomarker channel or surrogate channel as disclosed herein present in saliva.

For the fecal fat experiment, the processor can be configured with instructions to detect a change or lack of change in the fecal fat channel, or another biomarker channel or surrogate as disclosed herein present in fecal material.

Additional experiments can be conducted to measure one or more channels as described herein.

The processor can be configured with instructions to prompt the user for fasting tests such as blood fat level, triglyceride and glucose, or corresponding surrogates, for example.

The processor can be configured with instructions to prompt the user for a post-prandial test, such as blood fat level, triglyceride (2-4 hour post-prandial, TGpp), and glucose (1-1.5 hour post-prandial), or corresponding surrogates for example.

The processor can be configured with instruction to measure total protein channels and albumin channels, and globulins calculated from total protein and albumin ratios (TP-ALB), or corresponding surrogates, for example.

The processor can be configured with instructions for the user to conduct an experiment for an appropriate amount of time, such as 1 week to 8 weeks, for example 2 to 4 weeks, and in some embodiments 3 weeks.

The experiment may comprise a measurement to determine lowering triglycerides by eating walnuts (which may be detected by measuring the surrogate blood fat level, as described herein), lowering LDL by eating beta glucan, or consuming red yeast, for example. Similar lifestyle changes can be measured to determine increases or decreases in the channel corresponding to LDL, or ketones, for example.

The experiment may comprise measuring lower fasting glucose, fructosamine and HbA1c with an appropriate lifestyle change such as walking or adding chromium to the user's diet.

The processor can be configured with similar lifestyle changes to evaluate improvements in one or more inflammation channels as described herein.

The processor can be configured with instructions to allow the user to select a fecal fat experiment and evaluate changes in fecal fat in response to a lifestyle change as described herein.

In some embodiments, the spectrometer 100 comprises a broad spectrum light source to generate a plurality of wavelengths of light, a detector, and a wavelength selector coupled to the broad spectrum light source to selectively direct light toward the detector with the sample located between the wavelength selector and the detector. The wavelength selector may comprise one or more of a dispersive element, a prism, a grating, a digital mirror device (“DMD”), a diffractive optic, an interferometer, a Michelson interferometer, or an Etalon. In some embodiments, the spectrometer 100 comprises a digital micromechanical mirror optically coupled to the wavelength selector to selectively reflect the light from the wavelength selector to the detector.

In some embodiments, the detector comprises an indium gallium arsenide (InGaAs) detector. In some embodiments, the detector comprises a single element detector, while in other embodiments the detector comprises a plurality of detector elements. In some embodiments, the processor may take substantially continuous scans of a blood sample with a duty cycle within a range from about 10% to about 90% of a light source illuminating a detector of the spectrometer. In some embodiments, the spectrometer 100 comprises a receptacle to receive the sample holder (e.g., the sample holder 200 and/or the blood sample collector 300 illustrated in FIGS. 2A, 2B and 3) with the blood contained therein with an elongate axis of the sample holder oriented toward a vertical angle of inclination to separate the blood. In some embodiments, a number of spatially resolved sample locations along a height of the sample is within a range from about 2 to about 1000, and optionally within a range from about 5 to about 100.

In some embodiments, the plurality of wavelengths corresponds to a plurality of discretely resolved wavelength bands within a range from about 25 to about 1000 discretely resolved wavelength bands and the plurality of successive measurements is within a range from about 2 to about 1000 successive measurements. In some embodiments, the plurality of discretely resolved wavelength bands is within a range from about 50 to about 200 and the plurality of successive measurements is within a range from about 5 to about 200 successive measurements.

In some embodiments, the plurality of discretely resolved wavelength bands comprises a plurality of wavelength bands within a range from about 1350 nm to about 2500 nm. In some embodiments, the range can be from about 1600 nm to 2400 nm.

In some embodiments, the spectrometer comprises a maximum dimension of 170 mm and optionally wherein the spectrometer comprises a length of no more than about 170 mm, a width of no more than about 75 mm, and a height of no more than about 100 mm, and optionally wherein the spectrometer comprises a length within a range from about 80 to about 170 mm, a width within a range from about 30 to about 75 mm and a height within a range from about 50 to about 100 mm and optionally wherein the spectrometer comprises a volume within a range from about 120,000 mm3 (0.12 liter) to about 1,275,000 mm3 (1.275 liter). Based on the teachings provided herein, a person of ordinary skill in the art can decrease the dimensions with optics of decreased sizes and focal lengths, for example.

In some embodiments, the sample holder comprises an elongate channel. In this regard, the spectrometer 100 spectrometer may be configured to receive the sample holder and align the elongate channel of the sample holder 200 along a substantially vertical direction to separate the blood into the plurality of components along the elongate channel. The substantially vertical direction may comprise an angle within about 20 degrees of vertical. In this regard, a DMD of the spectrometer 100 and the processor may be configured to selectively scan a first region of the sample holder comprising a first component (e.g., blood plasma), and to selectively scan a second region of the sample holder comprising a second component (e.g., hematocrit). In some embodiments, the processor may be configured with instructions to determine an amount of time for the sample to separate into the first and second components.

In some embodiments, the processor directs substantially continuous scans of the sample with a duty cycle within a range from about 10% to about 90% of a light source illuminating a detector of the spectrometer.

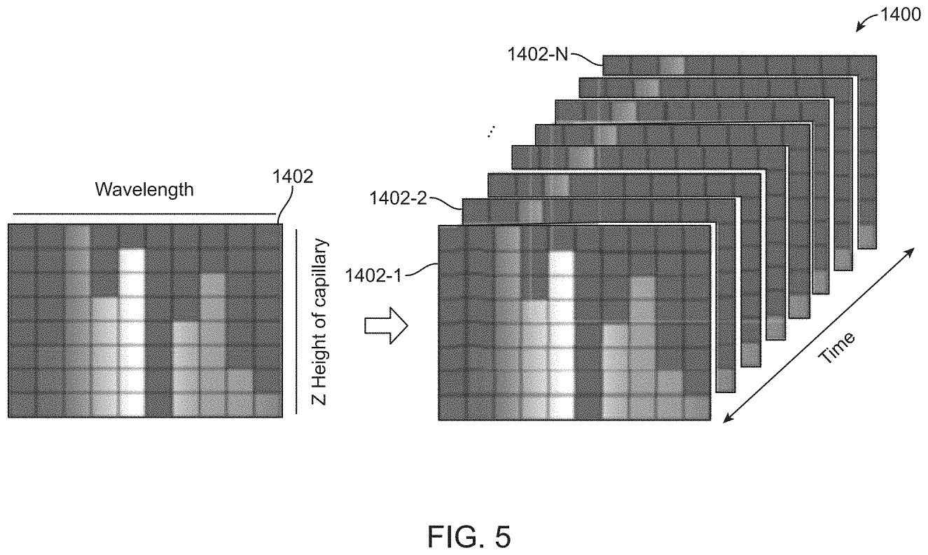

FIG. 5 shows spatially resolved spectral data 1400 over time from a spectrometer, in accordance with some embodiments. The spatially resolved spectral data 1400 may comprise a plurality of spatially resolved spectral measurements acquired at each of a plurality of times.

An exemplary plot 1402 of the spatially resolved spectral data 1400 shows the intensity at each of a plurality of wavelengths for each of a plurality of heights of the blood column in the sample holder, such as a capillary tube as described herein. The spectrometer can be configured to measure the spectra of the blood sample at a height Z in the column of blood as the sample separates. The height Z can range from about 1 mm to about 20 mm, for example from about 2 mm to about 10 mm. The number of spatially resolved samples locations along the height Z can range from about 2 to about 1000, for example within a range from about 5 to about 100. The mirror, phase modulator, grating or other wavelength selective component under computer control can be configured to measure the spectrum of the sample at each of the plurality of spatially resolved locations along the height of the sample.

The spectra are recorded for each location along the height of the column. The processor can be configured with instructions to measure each of a plurality of spatially resolved spectra, starting with a first spatially resolved spectral data corresponding to a first plot 1402-1 at a first time, followed by a second spatially resolved spectral data corresponding to a second plot 1402-2 at a second time, up to Nth spatially resolved spectral data acquired at Nth time and corresponding to an Nth plot 1402-N. The spatially resolved spectral data can be measured while the blood sample separates and stored by the processor as described herein.

The separation of the blood sample into red blood cells, plasma, white blood cells and platelets may comprise a gravimetric separation in which blood in the column at least partially separates into these components in response to gravity and different densities among the blood components as described herein. The timing of the separation and other spectral signals and the locations of these spectral signals in the separating blood column can provide useful information.

In some embodiments, the spatially resolved spectral data comprise hypercubes of spectral data comprising one or more of:

Quantitative molecular spectroscopic data of whole blood as it separates;

Mass separation rates, counts, heights, volume; or

Induced perturbations (including temperature, pressure, drying, coagulation agglutination, specialized chemical reaction).

The data may be labeled with real time information from lifestyle experiments such as food, exercise, supplements, etc.

The hypercubes of data may comprise vectors, in which each vector comprises a first dimension corresponding to spectral wavelength data, a second dimension corresponding to a spatial location of the spectral wavelength data, and a third dimension corresponding to time. For example, the first and second dimension may correspond to spatially resolved spectral data 1400 obtained at a time. The third dimension corresponding to time may comprise changes in the spatially resolved spectral data, for example changes to the spatially resolved spectral data as the blood separates.

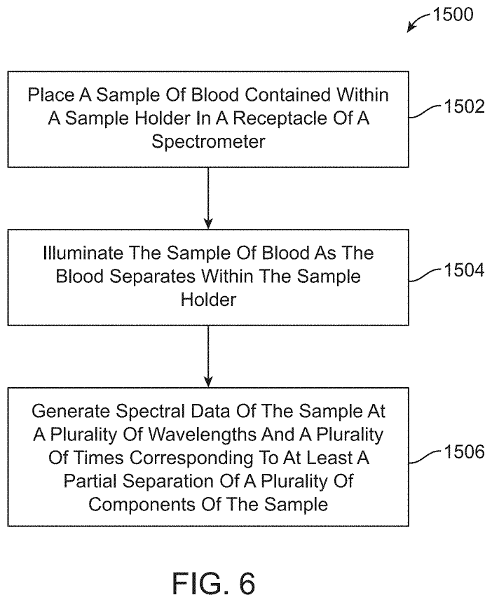

FIG. 6 shows a flowchart of an exemplary blood spectroscopy process 1500, in accordance with some embodiments. In this embodiment, a sample of blood contained within a sample holder (e.g., the sample holder 200) is placed in a receptacle of a spectrometer, such as the spectrometer 100 disclosed herein, in the process element 1502. The spectrometer may illuminate the sample of blood as the blood separates within the sample holder, in the process element 1504. For example, the blood may at least partially separate into a plurality of components, such as plasma, red blood cells, white blood cells, and the like as it is drawn into the sample holder. From there, the spectrometer and/or other processing system may generate spectral data of the sample at a plurality of wavelengths and a plurality of times corresponding to at least a partial separation of a plurality of components of the sample, in the process element 1506.

Examples of suitable biomarkers and chemometric analysis suitable for determining biomarkers are also described in PCT/US2016/026825, filed Apr. 8, 2016, entitled “METHOD AND APPARATUS FOR DETERMINING MARKERS OF HEALTH BY ANALYSIS OF BLOOD”, the entire disclosure of which is incorporated herein by reference. Although many chemometric approaches can be used, in some embodiments, a genetic algorithm is used to determine an amount of biomarker for a given biomarker channel. A plurality of spectral bins can be combined with appropriate weighting of each of the spectral channels in order to determine an amount of a biomarker. For example, approximately 300 to 400 spectral bins can be combined to determine a parameter related to health, such as blood pressure.

Additional approaches can be used with appropriate references and blood samples to determine the surrogates, markers and biomarkers as disclosed herein, such as Partial Least Squares (“PLS”) regression, and Null Augmented Regression (“NAR”). The NAR may comprise PLS coupled with Tikhonov Regularization that leverages the constant-analyte spectra of within-sample measurements of the calibration data. Random Forest Regression Tree (“RF/RT”) can also be employed. The RF/RT methodology can be used alternatively or in combination with a genetic algorithm as described in PCT/US2016/026825, the full disclosure of which has previously been incorporated by reference.

A channel of a surrogate or a biomarker may comprise the pure component spectrum of a surrogate or blood biomarker. The channel can be determined by calibrating the instrument using a set of labeled blood samples where the concentrations of the surrogate or biomarker are varied orthogonally to each other in a set of samples. This approach can be used to define the surrogate, marker and biomarker channels as disclosed herein, such as “blood fat level channel”, “glucose channel”, or “HDL channel.” In some embodiments, a channel is monitored for a change (or lack of change) during a lifestyle modification experiment as described herein. At the end of the experiment, which can last approximately 3 weeks, the channel is evaluated to determine whether the channel readout comprises a value higher or lower than where it started, or an unchanged value.

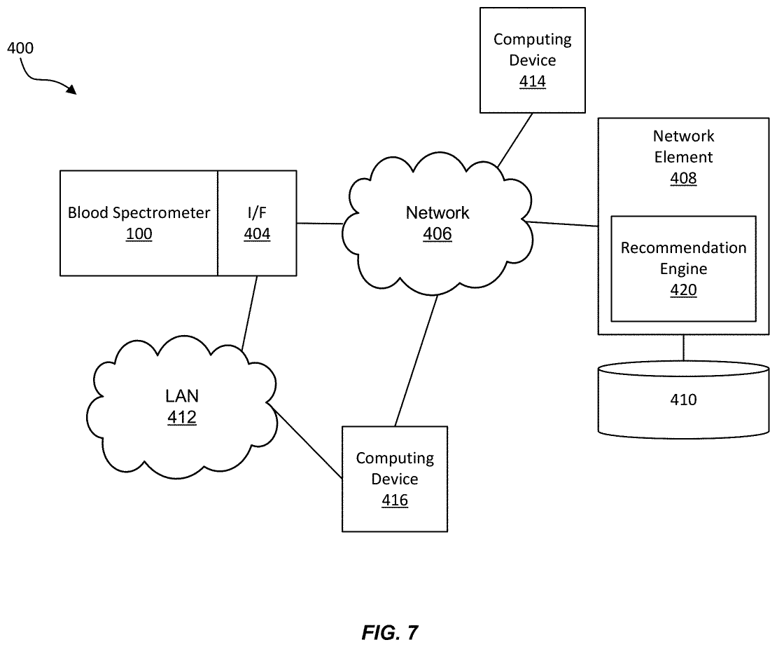

FIG. 7 shows a block diagram of an exemplary system 400 comprising a blood spectrometer 100 with network connectivity, in accordance with some embodiments. The blood spectrometer 100 may be configured with, or coupled to, a network interface (I/F) 404 that is communicatively coupled to a network 406 (e.g., the Internet) and/or a local area network (LAN) 412. For example, the blood spectrometer 100 may be configured to receive a sample of blood contained within a sample holder, such as the sample holder 200. The blood spectrometer 100 may illuminate the sample of blood as the blood at least partially separates within the sample holder. A processor operatively coupled to the blood spectrometer 100 and/or configured with the blood spectrometer 100 may be configured with instructions to generate spectral data of the sample at a plurality of wavelengths and a plurality of times, corresponding to at least partial separation of the sample of blood into a plurality of components of the sample.

When the blood spectrometer 100 detects the wavelength spectra of the various components of the blood 104, the blood spectrometer 100 may communicate the information pertaining to the wavelength spectra and/or the spectral data (e.g., spatially resolved spectral data acquired at a plurality of times) to the network 406 via the network I/F 404, which may in turn communicate the wavelength spectra and/or other spectral data to a network element 408 for subsequent processing. In this regard, the network element 408 may include, or be communicatively coupled to, a database 410 which may comprise various statistics and data pertaining to blood components that can be compared to and/or analyzed against the wavelength spectra of the blood 104. Alternatively, or additionally, the blood spectrometer 100 may include processing that communicates other relevant information pertaining to the blood 104 through the network 406 to the network element 408. Examples of the network element 408 include computer network servers, computing devices, communication routers, processors, and the like.

Also illustrated in this embodiment, is a computing device 414 that is communicatively coupled to the network 406. The computing device 414 may be used to perform analysis on the wavelength spectra and/or other spectral data of the blood from the blood spectrometer 100. In this regard, the computing device 414 may be in communication with the network element 408 to retrieve information pertaining to blood analysis such that a user of the computing device 414 (e.g., a medical professional, a trainer, or the like) can analyze the wavelength spectra from the blood spectrometer 100 and provide a diagnosis and/or other relevant information pertaining to the user's blood 104 to a user of the blood spectrometer 100. Examples of the computing device 414 include computers, smart phones, and the like, comprising various hardware (e.g., processors, memory, data storage devices, etc.), software, and/or firmware components for processing the wavelength spectra from the blood spectrometer 100.

In some embodiments where the blood spectrometer 100 is communicatively coupled to the LAN 412, the blood spectrometer 100 may be able to communicate wavelength spectra to a computing device 416. For example, the computing device 416 may also include computers, smart phones, and the like, comprising various hardware (e.g., processors, memory, data storage devices, etc.), software, and/or firmware components for processing the wavelength spectra and/or other spectral data from the blood spectrometer 100. In this regard, the computing device 416 may be that of the user using the blood spectrometer 100. For example, a user may draw his or her own blood 104 using the blood sample collector 300 described herein. The user may then input the sample of blood 104 into the blood spectrometer 100 to detect the various wavelength spectra of the components of the blood 104. The blood spectrometer 100 may then communicate the wavelength spectra to the user's computing device 416 such that the user may process the information and assess the user's own health.

Alternatively, or additionally, the computing device 416 may receive information pertaining to the user's blood spectroscopy from the network element 408 and/or the computing device 414. For example, once the user performs a spectroscopy on the user's blood sample via the blood spectrometer 100, the blood spectrometer 100 may convey the information to the network element 408 for analysis. The network element 408 may in turn produce results (blood fat level, LDL and HDL cholesterol levels, glucose levels, oxygen levels, hydration levels, sodium levels, iron levels, etc.) from the blood spectroscopy. The network element 408 may then return those results and/or any other relevant information pertaining to those results to the computing device 416 such that the user may view the results.

In some embodiments, the blood spectrometer 100, the computing devices 414 and 416, and the network element 408, either alone or in combination, may be configured with instructions (e.g., software components) that direct a processor to perform one or more analyses. For example, a processor configured with the blood spectrometer 100, the computing devices 414 and 416, and/or the network element 408 may measure two of more of a biomarker surrogate, a high density lipoprotein, a total cholesterol, a triglyceride or a glucose of the sample with a cross-validated standard errors of prediction (“CVSEP”) of no more than 12 mg/dL, 20 mg/dL, 40 mg/dL, 20 mg/dL, respectively, for each of the two of more of the high density lipoprotein, the total cholesterol, the triglyceride or the glucose of the sample, with the spectral data from the plurality of times corresponding to the at least partial separation of the sample. In some embodiments, the two or more comprises three or more of the total cholesterol, the triglyceride or the glucose of the sample with the cross-validated standard errors of prediction of no more than 12 mg/dL, 20 mg/dL, 40 mg/dL, 20 mg/dL. In some embodiments, the three or more comprises four or more of the total cholesterol components.

In some embodiments, the processor may measure the sample a plurality of times within a range from one minute to about 1 hour while the sample separates, and optionally within a range from about 2 minutes to about 30 minutes, and optionally within a range from about 5 minutes to about 15 minutes.

In some embodiments, the processor may measure one or more of a biomarker surrogate (such as blood fat level), a hormone (e.g., one or more of dehydroepiandrosterone (“DHEA”), Testosterone, Growth Hormone, Parathyroid Hormone, Estradiol, Progesterone, or Cortisol), a health and performance marker, the health and performance marker (e.g., one or more of Vitamin B12, PSA, Thyrogobulin, Troponin, IGF-1, Aldosterone, Prolactin, Creatine Kinase, Ferritin, Selenium, Homocystine, Copper, Ammonia, Folic Acid, AGE, or Cortisol), a metabolic marker (e.g., one or of Glucose, HbA1c, Glycated Albumin, Ketones, β-Hydroxybutyrate, Albumin, Total protein, BUN, Uric acid, Glutamate, GSH, Lactic Acid, CO2, pH, or Hydration), an immunology, inflammation and hematology marker (e.g., one or more of Fibrinogen, hsCRP, Globulins, Hematocrit, Hemoglobin, Erythrocyte sedimentation rate, Glutathione, Uric acid, Serum Amyloid A, Haptoglobin, WBC Count estimate, Transferrin saturation, Pyruvate, RBC count estimate, Platelet count estimate, Prothrombin time/INR, Interleukin-6), a cardiovascular marker (e.g., one or more of Cardiovascular blood fat level, total Cholesterol, HDL, LDL, Triglycerides, BNP, Apolipoprotein, or Average Blood Pressure), a marker of stress and toxins (e.g., one or more of blood fat level, Creatinine, Albumin, Carboxyhemoglobin, Ethanol, Carbon monoxide, Salicylates, Acetaminophen, or Caffeine).

In some embodiments, the blood spectrometer 100 comprises a broad spectrum light source to generate a plurality of wavelengths of light, a detector, and a wavelength selector coupled to the broad spectrum light source to selectively direct light toward the detector with the sample located between the wavelength selector and the detector. The wavelength selector may comprise one or more of a dispersive element, a prism, a grating, a DMD, a diffractive optic, an interferometer, a Michelson interferometer, or an Etalon. In some embodiments, the blood spectrometer 100 comprises a digital micromechanical mirror optically coupled to the wavelength selector to selectively reflect the light from the wavelength selector to the detector.

In some embodiments, the detector comprises an indium gallium arsenide (InGaAs) detector. In some embodiments, the detector comprises a single element detector, while in other embodiments the detector comprises a plurality of detector elements. In some embodiments, the processor may take substantially continuous scans of a blood sample with a duty cycle within a range from about 10% to about 90% of a light source illuminating a detector of the spectrometer. In some embodiments, the blood spectrometer 100 comprises a receptacle to receive the sample holder (e.g., the sample holder 200 and/or the blood sample collector 300 illustrated above) with the blood contained therein with an elongate axis of the sample holder oriented toward a vertical angle of inclination to separate the blood. In some embodiments, a number of spatially resolved sample locations along a height of the sample is within a range from about 2 to about 1000, and optionally within a range from about 5 to about 100.

In some embodiments, the plurality of wavelengths corresponds to a plurality of discretely resolved wavelength bands within a range from about 25 to about 1000 discretely resolved wavelength bands and the plurality of times is within a range from about 2 to about 1000, and optionally, wherein the plurality of discretely resolved wavelength bands is within a range from about 50 to about 200 and the plurality of times is within a range from about 50 to about 100.

In some embodiments, the plurality of discretely resolved wavelength bands comprises a plurality of wavelength bands within a range from about 1500 nm to about 2000 nm. In some embodiments, the range can be from about 1400 nm to 2400 nm.

In some embodiments, the blood spectrometer 100 comprises a maximum dimension of 170 mm and optionally the spectrometer comprises a length of no more than about 170 mm, a width of no more than about 75 mm, and a height of no more than about 100 mm, and optionally the spectrometer comprises a length within a range from about 80 to about 170 mm, a width within a range from about 30 to about 75 mm and a height within a range from about 50 to about 100 mm, and optionally the spectrometer comprises a volume within a range from about 120,000 mm3 (0.12 liter) to about 1,275,000 mm3 (1.275 liter). Based on the teachings provided herein, a person of ordinary skill in the art can decrease the dimensions with optics of decreased sizes and focal lengths, for example.

In some embodiments, the sample holder comprises an elongate channel. In this regard, the blood spectrometer 100 spectrometer may be configured to receive the sample holder and align the elongate channel of the sample holder 200 along a substantially vertical direction to separate the blood into the plurality of components along the elongate channel. The substantially vertical direction may comprise an angle within about 20 degrees of vertical. In this regard, a DMD of the blood spectrometer 100 and the processor may be configured to selectively scan a first region of the sample holder comprising a first component (e.g., blood plasma), and to selectively scan a second region of the sample holder comprising a second component (e.g., hematocrit). In some embodiments, the processor may be configured with instructions to determine an amount of time for the sample to separate into the first and second components.

In some embodiments, the processor directs substantially continuous scans of the sample with a duty cycle within a range from about 10% to about 90% of a light source illuminating a detector of the spectrometer.

The computing device may compare a first plurality of values of a surrogate for a biomarker or of the biomarker channels to a second plurality of corresponding values of the surrogate or the biomarker channels. Generally, the first plurality of values corresponds to a first measurement time and the second plurality of corresponding values corresponds to a second measurement time. Based on this information, the computing device may compute a change in at least one of the surrogate or biomarker channels and output that change to the user device.

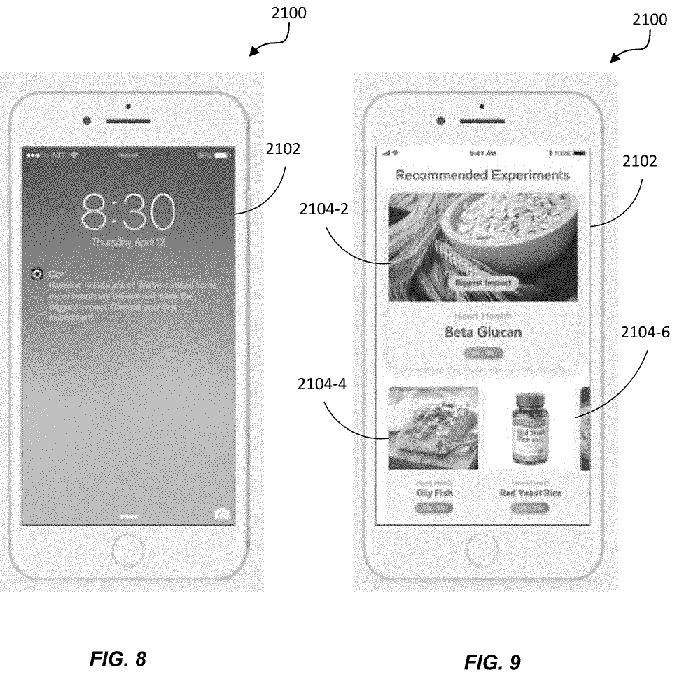

In some embodiments, a computing device of the system 400, such as the computing device 414 and/or the network element 408, may comprise a recommendation engine 420 that presents one or more lifestyle change experiments to a user via a graphical user interface of a user device, such as computing device 416. For example, FIG. 8 shows an exemplary user device 2100 (e.g., a smart phone) that may be configured with a software module or “app” that is operable to implement a graphical user interface (GUI) 2102 that prompts a user to perform one or more experiments, as directed by the recommendation engine 420. The user may view the experiments in the GUI 2102 as illustrated in FIG. 9.

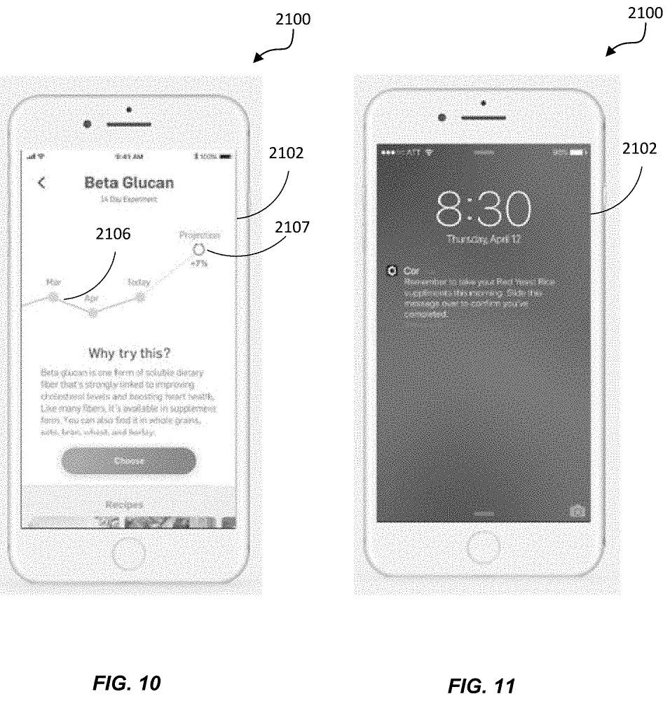

In this example, the GUI 2102 presents experiments pertaining to heart health by consuming beta glucan (experiment 2104-2), consuming oily fish (experiment 2104-4), or consuming red yeast rice (experiment 2104-6). The user may select the experiment through the GUI 2102 by tapping the experiment on the user device 2100. From there, the user device 2100 may convey the selection to the computing device. The recommendation engine 420 may in turn, based on the selected experiment, prompt or remind the user to perform a lifestyle change in accordance with the experiment. Afterwards, the recommendation engine 420 may prompt the user to take a blood sample for processing by the blood spectrometer 100. The blood spectrometer 100 may then convey the results of the blood spectroscopy (e.g., spectral data) through the network 406 to the computing device for further analysis. For example, the computing device may determine the effects of the experiment on the user, such as blood fat levels, LDL and HDL cholesterol levels, glucose levels, oxygen levels, hydration levels, sodium levels, iron levels, etc. Afterwards, the recommendation engine 420 may present the results of the selected experiment, based at least in part on the received spectroscopic data, via the GUI 2102 of the user device 2100. An example of such a presentation is illustrated in FIG. 10.

In FIG. 10, the user device 2100 exemplarily illustrates the results of the user's beta glucan experiment in the GUI 2102. For example, in the beta glucan experiment, the recommendation engine 420 may prompt the user a number of times to perform the experiment over the course of some duration (e.g., weeks or months). Each time, the recommendation engine 420 may direct the user through the software module on the user device 2100 to perform a blood spectroscopy on a new blood sample while the user is performing the experiment over the course of that duration. This information may be conveyed back to the computing device which updates the user's progress during the experiment and presents it as a line graph 2106 in the GUI 2102. The computing device may also compute and convey a projected result 2107 of the beta glucan experiment (e.g., the user selected experiment). The computing device may do the same for other experiments as well.

As mentioned, the recommendation engine 420 may prompt the user over time to perform the user selected experiments. FIG. 11 illustrates an embodiment where the recommendation engine 420 conveys, through the network 406 to the user device 2100, a prompt or reminder for the user to perform the red yeast rice test in the GUI 2102. For example, if the user has not performed the test within a certain amount of time, the recommendation engine 420 may message the user via device 2100 with a reminder to consume red yeast rice and/or perform another blood spectroscopy.

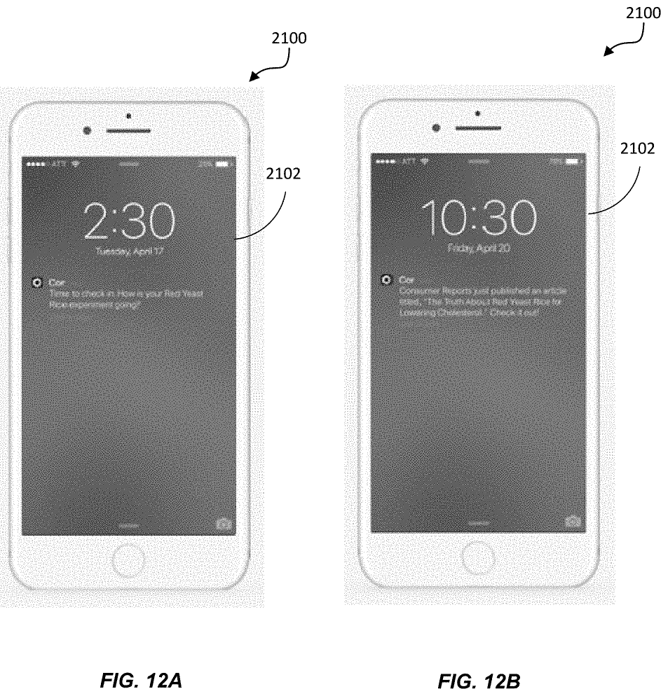

The recommendation engine 420 may also periodically send messages to the user device 2100 to inform and/or query the user. For example, FIG. 12A shows the GUI 2102 displaying a message from the recommendation engine 420 to the user asking how the red yeast rice experiment is going. FIG. 12B shows the GUI 2102 displaying a message from the recommendation engine 420 to the user with information pertaining to the benefits of red yeast rice.

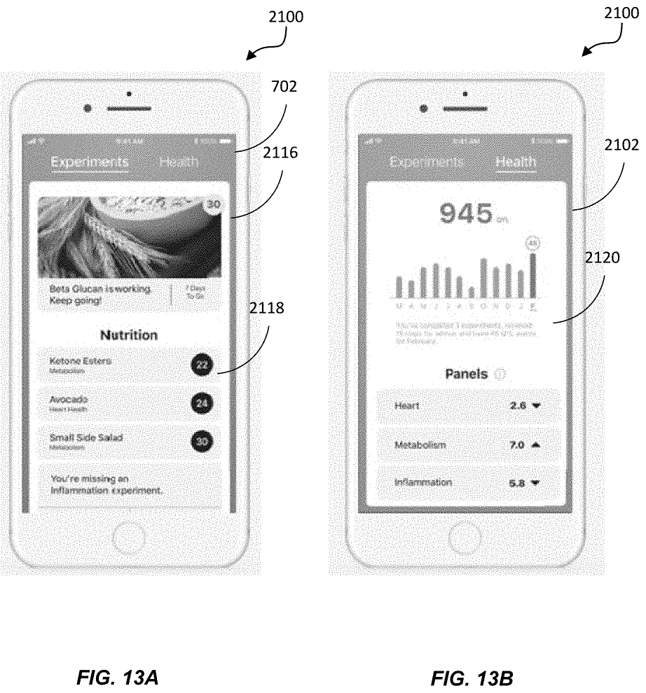

FIG. 13A shows an embodiment where the GUI 2102 of the user device 2100 provides encouragement to the user. In this example, the computing device may calculate the results 2116 of the beta glucan experiment, informing the user that the experiment is working and the recommendation engine 420 may provide a message to the user device 2100 illustrating such and encouraging the user to continue with the experiment. The recommendation engine 420 may also provide nutritional information 2118 pertaining to the experiment and/or other foods/edible substances that the user should consume. The recommendation engine 420 may also provide information pertaining to missing experiments. In this example, the GUI 2102 illustrates a message stating that the user is missing an inflammation experiment. FIG. 13B shows other results 2120 (e.g., heart condition, metabolism, inflammation, and the like) that the recommendation engine 420 conveys to the user device 2100 for display on the GUI 2102.

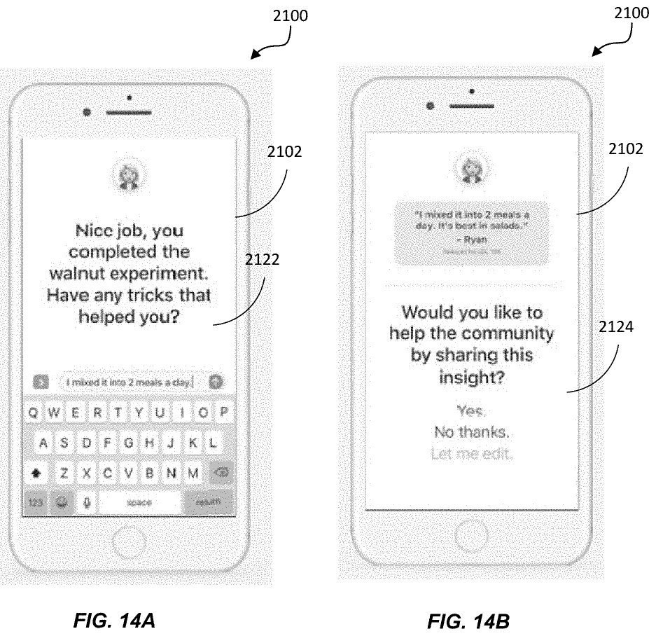

In some embodiments, the computing device may provide an interactive service between the user and the computing device. FIG. 14A illustrates the GUI 2102 of the user device 2100 with a message 2122 from the computing device querying the user for aids that assisted the user in a particular experiment. With this message, the computing device may provide a messaging field (e.g., short messaging service or “SMS”) through which the user can reply. The computing device may convey this information to a service representative and/or automatically parse words from the message of the user to select an appropriate response 2124 as shown in FIG. 14B. For example, the computing device may query the user as to whether the user would like to share the user's insights and tips that assisted the user in performing the walnut consumption or other experiment. From there, the user may select an appropriate automatic reply.

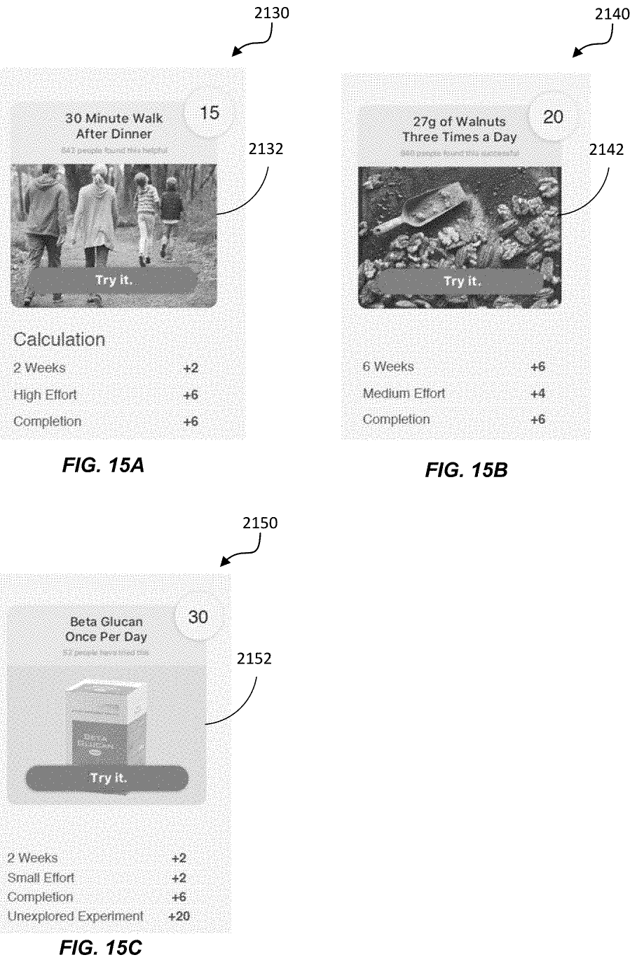

In some instances (e.g., where the user wishes to share information), the system 400 and its associated computing devices may implement a social networking platform. For example, if the user shares information to the network element 408, the network element 408 may share this information with other users communicating with the network element 408. In this regard, the network element 408 may compile statistics of other users and provide messages that may assist the user of the user device 2100. FIGS. 15A-15C shows various messages and information that may be displayed to the user illustrating experiments performed by other users. For example, message 2130 generated by the network element 408, in FIG. 15A, shows an experiment of a 30-minute walk after dinner that 642 people found helpful. Thus, the network element 408 may compile the information from the other users to deliver the message 2130 to the GUI 2102 of the user device 2100. And, in this message, the network element 408 may provide a link 2132 for the user of the user device 2100 to select. Such a selection may result in a message from the network element 408 to provide direction to the user to complete the experiment. FIG. 15B shows a similar message 2140 having the user consume 27 grams of walnuts three times a day with a selection link 2142. And, FIG. 15C shows a similar message 2150 having the user consume beta glucan once per day with a selection link 2152. Of course, these messages and the associated information may be implemented in a variety of ways.

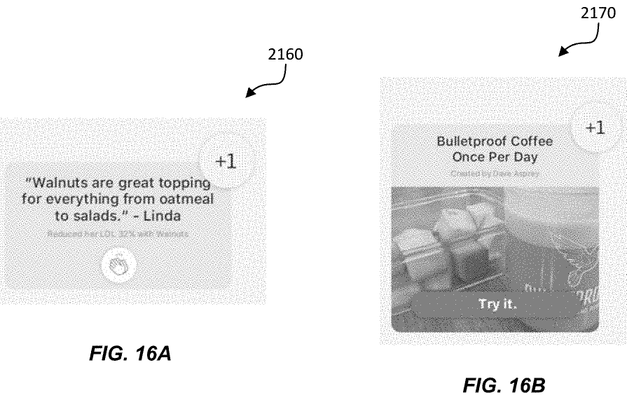

Additionally, other users may connect with the user of the user device 2100. For example, other users may send messages to the user of the user device 2100 through the social networking platform implemented by the system 400. In this regard, other users may send messages regarding certain results they achieved during experiments, provide experiment recommendations or words of encouragement, and the like. FIG. 16A shows a message 2160 conveyed from another user to the user of the user device 2100 stating how walnuts of the walnut experiment may be preferably consumed. And, FIG. 16B shows a message 2170 from a user encouraging the user of the user device 2100 to consume a certain type of coffee. These messages and/or results from other users may be obtained from their individual blood spectroscopies of their experiments that have been conveyed to the network element 408. For example, other users connected to the network element 408 may perform experiments that are similar to those performed by the user of the user device 2100. These other users may derive results from their individual blood spectroscopies involved in those experiments such that the results of those blood spectroscopies may be conveyed to the network element 408 for storage within the database 410.

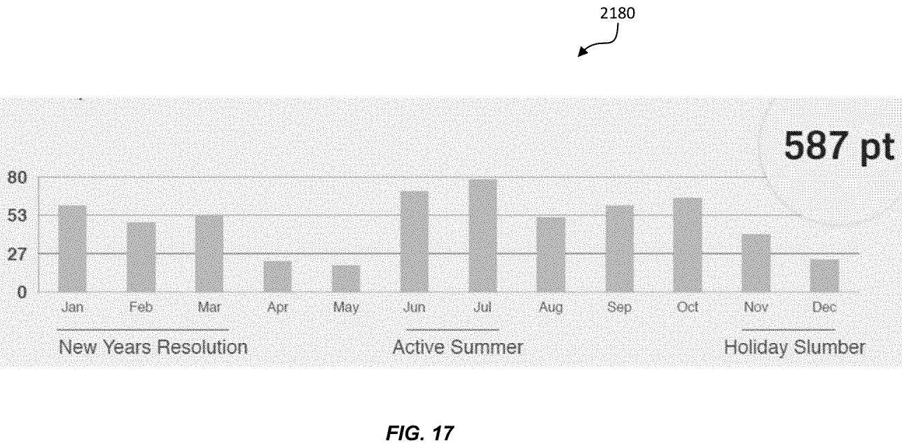

FIG. 17 shows a graph 2180 of a user's progress over the course of a year, in one exemplary embodiment. In this embodiment, the network element 408 may compute a score for each month the user of the user device 2100 is engaging in experiments and attempting to improve the user's biomarkers or biomarker surrogates through the experiments. Then, over the course the year, the network element 408 may compute an overall score for the user which may be shared with the user's social network. In this regard, the social media platform provided by the network element 408 may provide a means for encouraging a competition between users in the user social network. For example, in this embodiment, the user of the user device 2100 has an overall score 587 points for the course of a year. Other users may have higher or lower scores and may encourage each other to obtain better scores and improve their overall health.

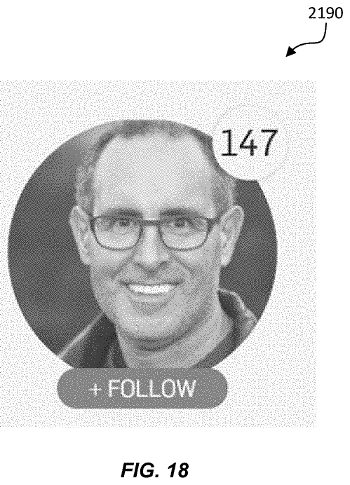

FIG. 18 shows an exemplary social networking aspect of the system 400. In this embodiment, the user 2190 has a network of 147 followers, of which one may be the user of the user device 2100. In this regard, the user of the user device 2100 and the user 2190 may communicate with one another to encourage each other and/or share health information pertaining to their respective blood spectroscopies, experiments performed, health scores, and the like.

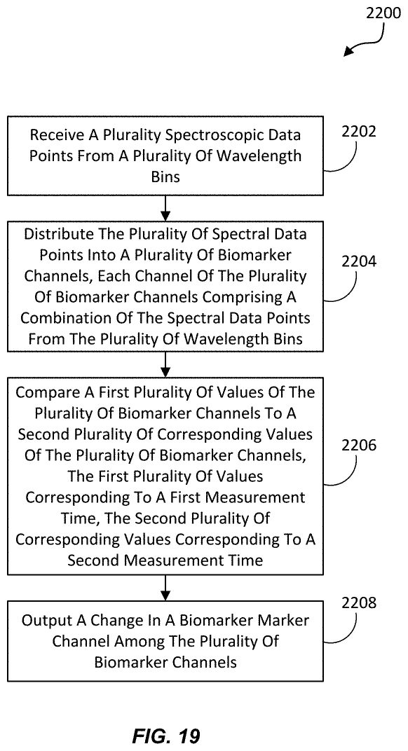

FIG. 19 shows a flowchart of an exemplary process 2200, in accordance with some embodiments. In this embodiment, a computing device, such as the computing device 414 and/or the network element 408 of FIG. 7, is configured to receive a plurality of spectroscopic data points from a plurality of wavelength bins, in the process element 2202. For example, the blood spectrometer 100 may generate spectral data pertaining to a user's blood sample. The blood spectrometer 100 may communicate that spectral data to the computing device for analysis. In this regard, the computing device may distribute the spectral data points into a plurality of biomarker or biomarker surrogate channels, in the process element 2204. Generally, each channel of the biomarker or biomarker surrogate channels comprises a combination of the spectral data points from the plurality of wavelength bins.

The computing device may compare a first plurality of values of the biomarker or biomarker surrogate channels to a second plurality of corresponding values of the channels, in the process element 2206. Generally, the first plurality of values corresponds to a first measurement time and the second plurality of corresponding values corresponds to a second measurement time. Based on this information, the computing device may compute a change in at least one of the biomarker or biomarker surrogate channels and output that change to the user device, in the process element 2208.

The comparison of the biomarkers or biomarker surrogates can have the benefit of showing how an experiment is changing the user's biomarkers or biomarker surrogates, and these can be presented to the user as described herein. Although the amount of biomarker or surrogate can be quantitative, the presently disclosed methods and apparatus can determine a change in a biomarker or surrogate in response to a user experiment, which can provide useful information to the user.

Experiments can be conducted to show the accuracy of the measurements obtained with spectrometers and blood samples as described herein. The data can be analyzed by chemometric methods and a N-fold “subject” out cross validation v. mean of all labs approach and compared with reference samples obtained from the American College of Physicians. Chemometric methods can be used to define the biomarker or surrogate channels based on weights combinations of spectral bins, such as Partial Least Squares (“PLS”) regression, and Null Augmented Regression (“NAR”). The NAR may include PLS coupled with Tikhonov Regularization that leverages the constant-analyte spectra of within-sample measurements of the calibration data. Random Forest Regression Tree (“RF/RT”) may also be employed.

Examples of suitable biomarkers and chemometric analysis suitable for determining biomarkers are described in PCT/US2016/026825, filed Apr. 8, 2016, entitled “METHOD AND APPARATUS FOR DETERMINING MARKERS OF HEALTH BY ANALYSIS OF BLOOD”, the entire disclosure of which has been previously incorporated herein by reference. Although many chemometric approaches can be used, in some embodiments, a genetic algorithm is used to determine an amount of biomarker for a given biomarker channel. A plurality of spectral bins can be combined with appropriate weighting of each of the spectral channels in order to an amount of a biomarker. For example, approximately 300 to 400 spectral bins can be combined to determine a parameter related to health, such as blood pressure.

The methods and apparatus as described herein can be configured with instructions to provide augmentation of the calibration space. While the calibration space augmentation can be performed in one or more of many ways with the factors and functions methods as described herein, the calibration space augmentation may comprise one or more of an augmented classical least squares of the calibration space data, an augmented partial least square of the calibration space data, or a multivariate curve resolution of the calibration space data. An iterative fit can be performed to linearly independent spectral data sets, for example. A spectral signature can be developed for one or more of the calibration space data or the blood sample data, for example. The spectral signature of the calibration space data can be used for later analysis of the blood sample as described herein, for example with one or more of partial least squares, augmented classical least squares, multivariate curve resolution, or other chemometric approach as described herein, for example.

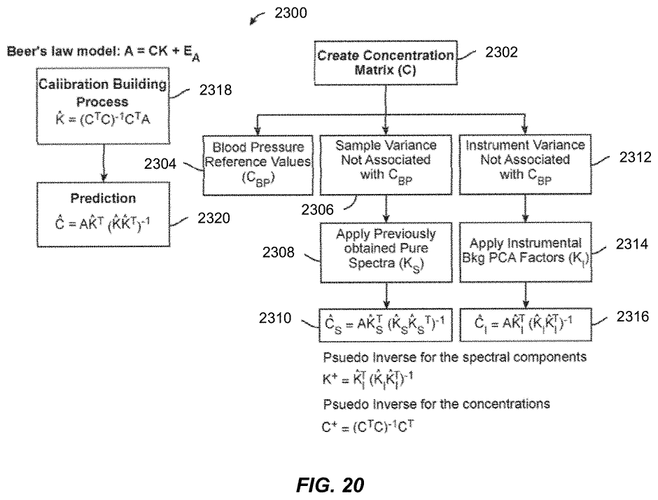

FIG. 20 shows a method 2300 of spectral data analysis suitable for incorporation with embodiments as disclosed herein. The analysis of spectral channels can be configured in many ways to determine the spectral bins that can be used to define a channel of a marker such as a biomarker or biomarker surrogate.

The marker, surrogate and biomarker channels as disclosed herein can be determined in many ways, and the following is provided as an example of a blood pressure channel suitable for incorporation with embodiments as disclosed herein. The approach described with reference to FIG. 20 is similar to the one described in PCT/US2016/026825, filed Apr. 8, 2016, entitled “METHOD AND APPARATUS FOR DETERMINING MARKERS OF HEALTH BY ANALYSIS OF BLOOD”, the full disclosure of which has previously been incorporated by reference.

A variant of Classical Least Squares (CLS) may be used to build calibration models and predict blood pressure values or other markers as described herein based on red blood cell spectra. This CLS variant has been referred to as Augmented CLS and can be performed during the prediction process. CLS assumes Beer's law behavior (A=CK+E_{A}), where A is the absorbance spectra, C is a matrix of concentrations, K is the pure component spectra and EA are the spectral residuals (i.e., anything unmodelled by a linear combination of C and K). Red blood cell spectra obtained using a measurement apparatus as described herein can be converted to absorbance by taking the minus Log₁₀ of the ratio of the red blood cell spectra to a close-in-time instrumental background spectrum. Since CLS tries to minimize E_{A}, all sources of spectral variation should be modelled through the concentrations (C) and the pure component spectra (K) in order to produce accurate resultant estimates. The pure component spectrum (K) of an analyte of interest is usually already known; therefore, augmentation usually occurs in the prediction process (solving for C). To prevent aberrant spectral variation (i.e., spectral variation not associated with the analyte of interest) from affecting the CLS model, the model may be proactively augmented with spectral component(s) associated with these aberrations, so that better estimates of the concentration of the analyte of interest can be obtained. The augmentation process may be applied during the calibration process, in order to get an accurate estimate of the pure spectral component associated with blood pressure, for example.

At step 2302, a concentration matrix C is created to obtain the pure spectral component of blood pressure or other marker or surrogate, as disclosed herein. This concentration matrix can be composed of blood pressure reference measurements (C_{B}p), concentrations associated with spectral variance during the measurement of the red blood cell samples but not associated with the red blood cells (Cs), and concentrations associated with spectral variance of the instrument (Ci). Concentrations CBP, CS, and Ci can be combined into one concentration matrix C and used to estimate the pure spectral components that can be used for later predictions.

At step 2304, the blood pressure reference values (CBP) or other marker or surrogate, as disclosed herein, are obtained. The blood pressure reference values CBP may comprise the mean of the blood pressures acquired over a period of time from a subject, to ensure the best estimate of the actual sustained blood pressures from the subject.

At step 2306, the concentrations associated with spectral variance during the measurement of the red blood cell samples (Cs) are obtained.

At step 2308, previously obtained pure spectral components (Ks) are applied.

Spectral components Ks may comprise spectral components of water, red blood cells, and spectral variation associated with a process applied to the red blood cells, such as gravimetric separation, as disclosed herein.

At step 2310, the concentrations Cs are estimated using CLS, from the pseudo inverse of the previously obtained pure spectral components Ks and the absorbance spectra A. The pseudo inverse K⁺ of the spectral components Ks can be obtained using the equation:

K⁺= [symbol] )⁻¹,

where [symbol] is the transpose of the matrix [symbol] .

At step 2312, the concentrations associated with the instrument variation (Ci) are obtained.

At step 2314, instrumental background spectra (Bkg) are applied. Background spectra Bkg may be taken during the entire period of absorbance spectra (A) data collection. These background spectra can comprise measurements of air (no sample in the sample compartment of an instrument), or measurements of a sample that most spectrally resembles the sample of interest but is not the actual sample of interest (e.g., water or saline). These background spectra can be decomposed into spectral factors or components—by using Principal Component Analysis (PCA), for example. The number of these spectral components—can be varied, such that only the largest sources of spectral variance are explained by these spectral components —.

At step 2316, the concentrations associated with the instrument variation Ci are estimated using CLS, from the pseudo inverse of the instrument variation spectral components Ki and the absorbance spectra A. The pseudo inverse K⁺ of the spectral components Kᵢ can be obtained using the equation:

K⁺= [symbol] )⁻¹,

where [symbol] is the transpose the matrix [symbol] .

At step 2318, the calibration model is built by using a CLS calculation to obtain the pure component spectra K of which the component of interest resides, from the pseudo inverse of the concentration matrix C and absorbance spectra. The pseudo inverse C⁺ of the concentrations C can be obtained using the equation C⁺=(C^{T}C)⁻¹ C^{T}, where C^{T} is the transpose the matrix C. The spectral component of interest can be, for example, the component associated with blood pressure or other marker or surrogate for a biomarker, as disclosed herein.

At step 2320, the concentration C of the component of interest is predicted using traditional CLS, from the pseudo inverse of the pure component spectra K and the absorbance spectra A. The pseudo inverse K⁺ of the spectral components K can be obtained using the equation:

K⁺= [symbol] )⁻¹,

where [symbol] is the transpose of the matrix [symbol] K.
The concentration C can be, for example, the blood pressure level or other marker or biomarker surrogate level, as disclosed herein. Using this prediction model, blood pressure or other marker or biomarker surrogate may be predicted using spectral data of blood samples acquired in the future by using traditional or augmented CLS methods.

In some embodiments, method 2300 discloses a method of predicting blood pressure from spectroscopic data from blood samples. A person of ordinary skill in the art will recognize many variations and modifications based on the disclosure provided herein. For example, some steps may be modified, some steps may be added or removed, some of the steps may comprise sub-steps, and many of the steps can be repeated.

The processor as described herein can be programmed with one or more instructions to perform one or more of the steps of the method 2300 of predicting blood pressure or other marker or biomarker surrogate, as disclosed herein using blood spectroscopic measurements. Therefore, the above steps are provided as an example of a method of measuring blood pressure of the subject in accordance with embodiments.

The methods of sample measurement and analysis as described herein may be optimized using computational algorithms. For example, one or more steps of the methods described herein involving the selection of a parameter may be optimized using a genetic algorithm. A genetic algorithm generally comprises a family of evolutionary search procedures that are based upon mechanisms of natural selection and genetics. A genetic algorithm may apply principles of survival of the fittest to solve general optimization problems.

A genetic algorithm may be used to optimize one or more steps of spectral data analysis as described herein. For example, a genetic algorithm may be applied to select a subset of wavelengths or frequencies of sample spectra to use in generating a calibration model to predict blood pressure or other marker or biomarker surrogate, as disclosed herein from red blood cell spectra. A sample spectrum usually comprises a plurality of measurements at a plurality of frequencies, wherein the plurality may comprise hundreds or thousands of data points. Therefore, selecting a subset of frequencies that are most relevant for predicting blood pressure (or other marker or surrogate, as disclosed herein) in building the calibration model can enhance the accuracy or predictiveness of the generated calibration model, as well as reduce the computational burden in building the calibration model and generating predictions.

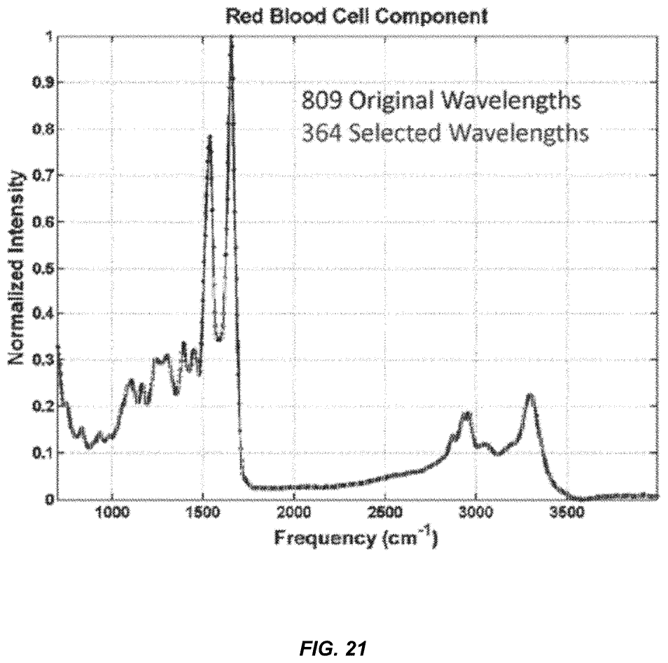

FIG. 21 shows the final selection of wavelengths of a red blood cell spectrum, optimized using a genetic algorithm procedure for a marker such as blood pressure, suitable for incorporation with the present disclosure. The original spectrum contained data points at 809 wavelength bands or frequencies. Using the genetic algorithm, 364 wavelengths were selected to be used in building the calibration model and generation of predicted blood pressures from the calibration model. The 364 selected wavelength bands were identified from generation of 19 wavelength strings. Some of the wavelength bands most consistently identified as important for the prediction of blood pressure included wavelengths of about 1950 to about 2000 cm⁻¹, which can contain a transition metal carbonyl band potentially indicative of the formation of a spectrin-hemoglobin complex, linked to the rigidity of the red blood cell membrane.

The measured spectral intensities of the 364 discrete wavelength bands can be combined with appropriate weighting of the genetic algorithm to define a blood pressure channel. In accordance with the present disclosure, changes to the blood pressure channel can be measured in response to a lifestyle or other change, and the change presented to a user or other users as disclosed herein.

Although FIG. 21 shows wavelength bands suitable for determining a blood pressure marker, the approach for determining a blood pressure channel can be used for any of the markers, biomarker surrogates, and biomarkers, as disclosed herein. A person of ordinary skill in the art can conduct similar experiments do determine the appropriate combinations and weights for the measured wavelengths bands to define a channel for a specific marker as disclosed herein. The changes in the channel can be monitored as disclosed herein.

As described, the system and methods disclosed herein may be used to measure a level or change in a marker or biomarker in a person's blood and use that information to assist the person to make beneficial changes to their nutrition, exercise, or lifestyle. In addition, the system and methods may be used to measure a surrogate for a biomarker and use information regarding the level or variance of the surrogate to recommend changes to the person's nutrition, exercise regimen, or lifestyle. In some embodiments, the measurement(s) of the surrogate for the biomarker may be used to provide feedback to a person regarding an “experiment” they are conducting to improve their health.

In some embodiments, a surrogate for a biomarker refers to a measurable quantity that is correlated to a biomarker. For example, in some embodiments, the surrogate comprises the level of fat in a person's blood or blood plasma, and the biomarker to which it is correlated is a triglyceride level. Thus, in some embodiments, by making spectroscopic measurements of a person's blood sample at different times, the fat level in their blood may be determined at those times and the variance in fat level may be determined during a time interval. Based on the variance in the fat level, the variance in the person's triglyceride level during the time interval is inferred. Because the variance, and in some cases the level of, triglycerides in a person's blood can be an indicator of health, the fat levels in a person's blood at various times or with reference to various activities (such as eating or exercising) can provide information that can be used to suggest ways for the person to improve their health.

Research performed in connection with the development of the system and methods described herein has indicated that the variance in the level of fat in a person's blood plasma is strongly correlated with changes in the triglyceride level in the person's blood. This allows a spectroscopic measurement of the fat level in blood to be used as a substitute for measuring triglyceride. Because of the significance of triglyceride levels and their variation to health (e.g., as a marker for inflammation due to diet, stress, and other factors that can impact health), the measurement of fat levels can provide assistance to a person as they attempt to modify their diet or exercise regimen to improve their health.

As an example, the system and methods described may be used to monitor a spike in triglyceride levels (as inferred from measurements of the surrogate fat level) as a result of eating or eating specific foods. This may be helpful to recommending dietary changes or lifestyle changes to a person so as to avoid or reduce such a spike. Further, monitoring fat levels over a time interval may provide data that can be used to develop a model of the person's response to fasting, diet, exercise, or other behaviors. The model may then be used to generate recommendations of activities to participate in, not participate in, or delay in order to minimize the negative impact of an elevated triglyceride level.

Still further, the reliability of the detection of a possible triglyceride spike may be impacted by how recently a person has eaten. In order to prevent this problem, in some embodiments, a measured level of glucose or protein, or an elevated level of glucose or protein may be used to advise a person to provide a blood sample at a different time.

In some embodiments, information regarding a person's activities in a time interval prior to their providing a blood sample for analysis may be collected and used to associate changes in their diet, consumption of a specific foods, or engaging in a specific activity with levels of a biomarker surrogate in their blood. This may allow a model to better characterize the person's physiology and improve recommended changes or “experiments” for that person. Further, collecting such data for a group of people may provide ways to compare a person's measurement(s) to that of a similarly situated group and thereby improve recommendations for the person.

In some embodiments, the system and methods may be configured to generate an alert if the level of a biomarker surrogate exceeds a threshold value or undergoes a significant variance during a time interval. This can inform a user of an association between their behavior or activity and the level or change in level of the measured surrogate.

While the example of the fat level in blood serving as a surrogate for triglycerides has been discussed, the system and methods may be used to monitor other surrogates as well. For example, in some embodiments, the surrogate is a sedimentation rate and the biomarker is a triglyceride level. This is because an elevation in the triglyceride level increases the sedimentation rate, where that rate is used to improve decomposition of blood fats into triglycerides as opposed to other fats. Other combinations of surrogates and correlated biomarkers are also possible.

The system and methods described herein provide many benefits and advantages. These include one or more of the following: 1) requires a relatively small amount of blood, such that blood can be drawn with the smallest, most comfortable lancets, from a fingertip and eventually from alternate sites such as the forearm; 2) provides the ability to separate blood passively into cellular and plasma phases for analysis of both, for example no use of centrifuge to separate blood components and for example pure gravity-based separation; 3) a multiplexed method, such as the ability to get multiple “numbers” from each micro-drop of blood (in contrast, most in-home tests use chemical derivatization of a blood drop and can only provide one answer per blood drop, for example glucose not ketones, or ketones and not glucose); 4) reagentless, e.g. provides a simple low-cost in-home measurements system without use of a reagent; or 5) provides accuracy and precision, and frequent measurements can balance the accuracy and precision requirements where needed.

The described system and methods provide a relatively low-cost way of allowing a person to monitor certain biomarkers or biomarker surrogates in their blood, with samples provided using a simple finger prick device. Monitoring of the biomarkers or surrogates, along with information about the person's activities or behavior, can be used to create a model for how the person's metabolism and physiology respond to certain foods or activities. This model can be used to assist the person to understand how to make beneficial changes to their diet, exercise regimen, or other aspects of their lifestyle.

Although reference is made to changes in fat as a surrogate for triglycerides, the presently disclosed methods and apparatus can be used to determine surrogates for many biomarkers. The spectral binning and biomarker channels as described herein can be analyzed, e.g. mined, with one or more of deep learning, machine learning, convolutional neural networks or artificial intelligence to determine suitable spectral channels and combinations of spectral channels corresponding to surrogates for biomarkers. In some embodiments, the marker channel as described herein comprises a marker channel for a surrogate for a biomarker. Spectral data can be acquired from several blood samples and correlations determined for changes in surrogates and corresponding biomarkers. These surrogates corresponding to changes in biomarkers can be used to present data and suggested health experiments to a user as described herein.

In some embodiments, a metabolism experiment detects a change in a biomarker surrogate corresponding to one or more of the following channels: a level of fat in the blood sample, glucose, HbA1c (Glycated Hemoglobin), glycated albumin, ketones, β-hydroxybutyrate, albumin, total protein, blood urea nitrogen (BUN), uric acid, creatinine, glutamate, lactic acid (lactate), CO2 (bicarbonate), pH, sodium, magnesium, potassium, calcium, hydration, total body water (TBW), hematocrit, vitamin E, vitamin C, or vitamin A.

In some embodiments, a cardiovascular experiment detects a change in in a biomarker surrogate corresponding to one or more of the following channels: a level of fat in the blood sample, high density lipoprotein (HDL), low density lipoprotein (LDL), total cholesterol and other cholesterol ratios, apolipoprotein, or average blood pressure.

As detailed above, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions, including networked processors such as a server farm. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

The term “network element,” as used herein, generally represents any devices, systems, software, processor, or combinations thereof capable of providing communication through a network. Examples of such include network servers, computing devices, interfaces, databases, storage devices, communication interfaces, and the like.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. For example, one or more of the devices recited herein may receive image data of a sample to be transformed, transform the image data, output a result of the transformation to determine a 3D process, use the result of the transformation to perform the 3D process, and store the result of the transformation to produce an output image of the sample. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

The present disclosure includes the following numbered clauses:

Clause 1. A method comprising: receiving a blood sample; generating a plurality of spectroscopic data points from the blood sample using a spectroscopy device, wherein the plurality of spectroscopic data points comprises spectrometer data of the blood sample taken over a time interval and the spectrometer data comprises intensities from a plurality of wavelength bins; distributing the plurality of spectroscopic data points into a plurality of channels based on the plurality of wavelength bins, wherein each of the plurality of wavelength bins is associated with one or more of the plurality of channels and each of the plurality of channels comprises a combination of spectral measurement values from the plurality of wavelength bins; analyzing the plurality of channels for each channel of the plurality of channels to determine a value for a surrogate for a biomarker as a function of time during the time interval; determining if there is a significant change in the value of the surrogate over the time interval.

Clause 2. The method of clause 1, further comprising: correlating the value of the surrogate over the time interval to the biomarker; based on the correlation, determining whether to recommend a change to nutrition, exercise regimen, or lifestyle; and if a change is recommended, generating a user interface on a device to present the change to a user of the device.

Clause 3. The method of clause 1, wherein the spectroscopy device comprises a near infrared spectroscopy device.

Clause 4. The method of clause 1, wherein the surrogate comprises a blood fat level and the biomarker comprises a triglyceride level.

Clause 5. The method of clause 1, further comprising separating the blood sample into blood cells and blood plasma prior to generating the plurality of spectroscopic data points.

Clause 6. The method of clause 5, further comprising determining the value of the fat level in the blood plasma twice in a period of a day and based on a difference between those two measurements, determining whether to recommend a change to nutrition, exercise regimen, or lifestyle.

Clause 7. The method of clause 6, wherein the difference between the two measurements is used as an indicator of one or more of nutrition or stress.

Clause 8. The method of clause 1, further comprising measuring a glucose level in the blood sample, and using the glucose level to determine if the change in the level of the surrogate should be used to determine whether to recommend a change to nutrition, exercise regimen, or lifestyle.

Clause 9. The method of clause 1, further comprising measuring a protein level in the blood sample, and using the protein level to determine if the change in the level of the surrogate should be used to determine whether to recommend a change to nutrition, exercise regimen, or lifestyle.

Clause 10. The method of clause 8, wherein if the glucose level is used to determine that the change in the level of the surrogate should not be used to determine whether to recommend a change to nutrition, exercise regimen, or lifestyle, then the method further comprises generating a notification on the device that the value of the surrogate should be remeasured at a later time.

Clause 11. The method of clause 9, wherein if the protein level is used to determine that the change in the level of the surrogate should not be used to determine whether to recommend a change to nutrition, exercise regimen, or lifestyle, then the method further comprises generating a notification on the device that the value of the surrogate should be remeasured at a later time.

Clause 12. The method of clause 1, further comprising receiving information describing a current nutrition, exercise regimen, or lifestyle, and wherein the recommended change is to a characteristic of the current nutrition, exercise regimen, or lifestyle.

Clause 13. The method of clause 1, wherein the surrogate is a sedimentation rate and the biomarker is a triglyceride level.

Clause 14. The method of clause 1, further comprising receiving information regarding the time of receiving the blood sample and a person's intake of food or exercising and the time of that intake or exercising; and using the received information as training data for a model of the person's variation in the value of the surrogate over a period of time.

Clause 15. An apparatus comprising: a processor configured with instructions for receiving a blood sample; generating a plurality of spectroscopic data points from the blood sample using a spectroscopy device, wherein the plurality of spectroscopic data points comprises spectrometer data of the blood sample taken over a time interval and the spectrometer data comprises intensities from a plurality of wavelength bins; distributing the plurality of spectroscopic data points into a plurality of channels based on the plurality of wavelength bins, wherein each of the plurality of wavelength bins is associated with one or more of the plurality of channels and each of the plurality of channels comprises a combination of spectral measurement values from the plurality of wavelength bins; analyzing the plurality of channels for each channel of the plurality of channels to determine a value for a surrogate for a biomarker as a function of time during the time interval.

Clause 16. The apparatus of clause 15, wherein the processor is configured for: determining if there is a significant change in the value of the surrogate over the time interval; correlating the value of the surrogate over the time interval to the biomarker; based on the correlation, determining whether to recommend a change to nutrition, exercise regimen, or lifestyle; and if a change is recommended, generating a user interface on a device to present the change to a user of the device.

Clause 17. The apparatus of clause 15, wherein the spectroscopy device comprises a near infrared spectroscopy device.

Clause 18. The apparatus of clause 15, wherein the surrogate comprises a blood fat level and the biomarker comprises a triglyceride level.

Clause 19. The apparatus of clause 15, further comprising separating the blood sample into blood cells and blood plasma prior to generating the plurality of spectroscopic data points.

Clause 20. The apparatus of clause 19, further comprising determining the value of the fat level in the blood plasma twice in a period of a day and based on a difference between those two measurements, determining whether to recommend a change to nutrition, exercise regimen, or lifestyle.

Clause 21. The apparatus of clause 20, wherein the difference between the two measurements is used as an indicator of one or more of nutrition or stress.

Clause 22. The apparatus of clause 15, further comprising measuring a glucose level in the blood sample, and using the glucose level to determine if the change in the level of the surrogate should be used to determine whether to recommend a change to nutrition, exercise regimen, or lifestyle.

Clause 23. The apparatus of clause 15, further comprising measuring a protein level in the blood sample, and using the protein level to determine if the change in the level of the surrogate should be used to determine whether to recommend a change to nutrition, exercise regimen, or lifestyle.

Clause 24. The apparatus of clause 22, wherein if the glucose level is used to determine that the change in the level of the surrogate should not be used to determine whether to recommend a change to nutrition, exercise regimen, or lifestyle, then the method further comprises generating a notification on the device that the value of the surrogate should be remeasured at a later time.

Clause 25. The apparatus of clause 23, wherein if the protein level is used to determine that the change in the level of the surrogate should not be used to determine whether to recommend a change to nutrition, exercise regimen, or lifestyle, then the method further comprises generating a notification on the device that the value of the surrogate should be remeasured at a later time.

Clause 26. The apparatus of clause 15, further comprising receiving information describing a current nutrition, exercise regimen, or lifestyle, and wherein the recommended change is to a characteristic of the current nutrition, exercise regimen, or lifestyle.

Clause 27. The apparatus of clause 15, wherein the surrogate comprises a sedimentation rate and the biomarker comprises a triglyceride level.

Clause 28. The apparatus of clause 15, further comprising receiving information regarding the time of receiving the blood sample and a person's intake of food or exercising and the time of that intake or exercising; and using the received information as training data for a model of the person's variation in the value of the surrogate over a period of time.

Clause 29. A method comprising: obtaining a first sample of blood from a person at a first time; spectroscopically analyzing the first sample to determine a first value for a surrogate for a biomarker; obtaining a second sample of blood from the person at a second time; spectroscopically analyzing the second sample to determine a second value for the surrogate for the biomarker; determining a difference between the first value and the second value; and generating a user interface on a device to present the change to the person.

Clause 30. The method of clause 29, further comprising: correlating the difference to a change in a value of the biomarker during the time interval between the first time and the second time; and based on the correlation, determining whether to recommend a change to the person's nutrition, exercise regimen, or lifestyle.

Clause 31. The method of clause 29, wherein the spectroscopy device comprises a near infrared spectroscopy device.

Clause 32. The method of clause 29, wherein the surrogate comprises a blood fat level and the biomarker comprises a triglyceride level.

Clause 33. The method of clause 29, further comprising separating the first and second blood samples into blood cells and blood plasma prior to spectroscopically analyzing the blood samples.

Clause 34. The method of clause 33, further comprising determining the value of the fat level in the blood plasma twice in a period of a day and based on a difference between those two measurements, determining whether to recommend a change to the person's nutrition, exercise regimen, or lifestyle.

Clause 35. The method of clause 34, wherein the difference between the two measurements is used as an indicator of one or more of nutrition or stress.

Clause 36. The method of clause 29, further comprising measuring a glucose level in the blood sample, and using the glucose level to determine if the change in the level of the surrogate should be used to determine whether to recommend a change to the person's nutrition, exercise regimen, or lifestyle.

Clause 37. The method of clause 29, further comprising measuring a protein level in the blood sample, and using the protein level to determine if the change in the level of the surrogate should be used to determine whether to recommend a change to the person's nutrition, exercise regimen, or lifestyle.

Clause 38. The method of clause 36, wherein if the glucose level is used to determine that the change in the level of the surrogate should not be used to determine whether to recommend a change to nutrition, exercise regimen, or lifestyle, then the method further comprises generating a notification on the device that the value of the surrogate should be remeasured at a later time.

Clause 39. The method of clause 37, wherein if the protein level is used to determine that the change in the level of the surrogate should not be used to determine whether to recommend a change to nutrition, exercise regimen, or lifestyle, then the method further comprises generating a notification on the device that the value of the surrogate should be remeasured at a later time.

Clause 40. The method of clause 29, further comprising receiving information describing a current nutrition, exercise regimen, or lifestyle of the person, and wherein the recommended change is to a characteristic of the current nutrition, exercise regimen, or lifestyle.

Clause 41. The method of clause 29, wherein the surrogate is a sedimentation rate and the biomarker is a triglyceride level.

Clause 42. The method of clause 29, further comprising receiving information regarding the time of receiving the blood sample and a person's intake of food or exercising and the time of that intake or exercising; and using the received information as training data for a model of the person's variation in the value of the surrogate over a period of time.

Clause 43. The method of clause 42, wherein the model is used to recommend to the person when to provide a blood sample for analysis.

Clause 44. The method of clause 42, further comprising receiving from the person information regarding the food they consumed prior to providing a blood sample and determining how the value of the surrogate for the biomarker is affected by the food the person consumed.

Clause 45. The method of clause 44, further comprising generating a recommendation to the person regarding the food they consume in order to reduce the variation in the biomarker over a period of time.

Clause 46. An apparatus, comprising: a spectrometer configured to receive a sample of blood contained within a sample holder, to illuminate the sample of blood as the blood at least partially separates within the sample holder; a processor operatively coupled to the spectrometer, the processor configured with instructions to generate spectral data of the sample at a plurality of wavelengths and a plurality of times corresponding to at least partial separation of the sample of blood into a plurality of components of the sample; and determining a value for a surrogate for a biomarker from the plurality of components.

Clause 47. The apparatus of clause 46, wherein the processor is further configured with instructions to determine the value for the surrogate for a biomarker at two times during a time interval from the spectral data.

Clause 48. The apparatus of clause 47, wherein the surrogate comprises a blood fat level and the biomarker comprises a triglyceride level, and further, wherein the time interval is a day.

Clause 49. A method, comprising: placing a sample of blood contained within a sample holder in a receptacle of a spectrometer; illuminating the sample of blood as the blood separates within the sample holder; generating spectral data of the sample at a plurality of wavelengths and a plurality of times corresponding to at least a partial separation of the blood into a plurality of components of the sample; and determining a value for a surrogate for a biomarker based on the plurality of components.

Clause 50. The method of clause 49, further comprising determining the value for the surrogate for the biomarker at two times during a time interval from the spectral data.

Clause 51. The method of clause 50, wherein the surrogate is blood fat level and the biomarker comprises a triglyceride level, and further, wherein the time interval is a day.

Clause 52. A tangible medium configured with instructions for: receiving a plurality spectroscopic data points from a plurality of wavelength bins; distributing the plurality of spectral data points into a plurality of marker channels, each channel of the plurality of marker channels comprising a combination of the spectral data points from the plurality of wavelength bins; comparing a first plurality of values of the plurality of marker channels to a second plurality of corresponding values of the plurality of marker channels, the first plurality of values corresponding to a first measurement time, the second plurality of corresponding values corresponding to a second measurement time; and based on the comparison, outputting a change in a value for a surrogate for a biomarker to a user device.

Clause 53. The tangible medium of clause 52, wherein comparing the plurality of channels comprises comparing each value of the first plurality of values to a corresponding value of the second plurality of values.

Clause 54. The tangible medium of clause 52, wherein the plurality of channels comprises a vector, each value of the vector corresponding to a combination of the plurality of wavelength bins.

Clause 55. The tangible medium of clause 52, wherein the plurality of wavelength bins comprises wavelength bins spaced apart with non-overlapping wavelengths and wherein values of the plurality of channels are determined based on the plurality of wavelength bins comprising non-overlapping wavelengths.

Clause 56. The tangible medium of clause 52, wherein a change in each of the plurality of combination values from the first time to the second time is determined based on a change from the first time to the second time of said each of the plurality of channels.

Clause 57. The tangible medium of clause 52, wherein each of the plurality of channels comprises a weighted combination of spectral data from the plurality of wavelength bins.

Clause 58. The tangible medium of clause 52, wherein the plurality of wavelength bins comprises at least about 50 wavelength bins and wherein each of the plurality of channels comprises a combination values of the at least about 50 discrete wavelength bins.

Clause 59. The tangible medium of clause 52, wherein a portion of the plurality of channels consists of a same wavelength bin, and each channel of the portion comprises a different combination of the same wavelength bin.

Clause 60. A method comprising: receiving a plurality of spectroscopic data points, wherein the plurality of spectroscopic data points comprises spectrometer data of samples taken over a time interval and the spectrometer data comprises intensities from a plurality of wavelength bins; distributing the plurality of spectroscopic data points into a plurality of channels based on the plurality of wavelength bins, wherein each of the plurality of wavelength bins is associated with one or more of the plurality of channels and each of the plurality of channels comprises a combination of spectral measurement values from the plurality of wavelength bins; and analyzing the plurality of channels for each channel of the plurality of channels to detect a significant change in a value of a surrogate for a biomarker over the time interval.

Clause 61. The method of clause 60, further comprising monitoring a channel among the plurality of channels for a change in the channel.

Clause 62. The method of clause 61, wherein monitoring the channel for the change in the channel comprises: combining first measurement data from the plurality of wavelength bins associated with the channel from a first measurement to generate a first value of the channel; and combining second measurement data from the plurality of spectral channels associated with the channel from a first measurement to generate a second value of the measurement channel; comparing the first value of the measurement channel with the second value of the measurement channel to determine the change in the measurement channel.

Clause 63. The method of clause 60, wherein distributing the plurality of spectroscopic data points comprises: distributing the plurality of spectroscopic data points into the plurality of channels, wherein a value of the channel for each channel corresponds to intensity values of associated wavelength bins.

Clause 64. The method of clause 60, wherein analyzing the plurality of channels comprises: identifying for each channel of the plurality of channels an amount of change over the time interval.

Clause 65. The method of clause 60, wherein analyzing the plurality of channels comprises: generating a first value for each of the plurality of channels from first spectral data of the plurality of wavelength bins; generating a second value for each of the plurality of channels from second spectral data of the plurality of wavelength bins; determining a difference between the first value and the second value for each of the plurality of channels; and detecting the significant change based on the difference above a threshold for one or more of said each of the plurality of channels.

Clause 66. The method of clause 65, wherein the first values of the plurality of channels comprise control values.

Clause 67. The method of clause 60, wherein the plurality of spectroscopic data points corresponds to periodic blood samples taken over the time interval and measured by a spectrometer as part of a health experiment.

Clause 68. The method of clause 67, wherein the periodic blood samples correspond to a plurality of users.

Clause 69. The method of clause 68, wherein the time interval corresponds to a period of time during which each of the plurality of users implement a lifestyle change as part of the health experiment.

Clause 70. The method of clause 67, wherein the spectrometer is configured for reagentless whole blood spectroscopy.

Clause 71. The method of clause 60, wherein the spectral datapoints comprise spectral measurements from a whole blood sample and optionally wherein the time interval corresponds to a first a spectral measurement of a first blood sample and a second spectral measurement of a second blood sample.

Clause 72. The method of clause 60, wherein the plurality of channels corresponds to at least 200 resolved wavelength bins and the plurality of channels comprises at least 8 channels.

Clause 73. A method comprising: presenting at least one suggested experiment regarding nutrition, an exercise regimen, or lifestyle to a user via a graphical user interface of a user device; receiving a selection of the suggested experiment in a computing device; prompting, from the computing device and based on the selected experiment, a reminder to the user to perform a change to their nutrition, an exercise regimen, or lifestyle; prompting, from the computing device, the user to take a blood sample; processing, in the computing device, spectroscopic data corresponding to the blood sample; and presenting results of the selected experiment based at least on the received spectroscopic data via the graphical user interface of the user device, wherein the results comprise a change in a value of a surrogate for a biomarker over a time interval.

Clause 74. The method of clause 73, wherein the experiment comprises one or more of a metabolism experiment, a cardiovascular health experiment, an inflammation and immune function experiment, hematologic function experiment, a toxin experiment, a stress experiment, a saliva experiment, or a fecal fat experiment.

Clause 75. The method of clause 74, wherein the metabolism experiment detects a change in a biomarker surrogate corresponding to one or more of the following channels: a level of fat in the blood sample, glucose, HbA1c (Glycated Hemoglobin), glycated albumin, ketones, β-hydroxybutyrate, albumin, total protein, blood urea nitrogen (BUN), uric acid, creatinine, glutamate, lactic acid (lactate), CO2 (bicarbonate), pH, sodium, magnesium, potassium, calcium, hydration, total body water (TBW), hematocrit, vitamin E, vitamin C, or vitamin A.

Clause 76. The method of clause 74, wherein the cardiovascular experiment detects a change in in a biomarker surrogate corresponding to one or more of the following channels: a level of fat in the blood sample, high density lipoprotein (HDL), low density lipoprotein (LDL), total cholesterol and other cholesterol ratios, apolipoprotein, or average blood pressure.

Clause 77. The method of clause 74, wherein the fecal fat experiment detects a change in a fecal fat channel.

Clause 78. The method of clause 73, wherein prompting the user includes periodic prompts for the user to perform the lifestyle change in accordance with the experiment.

Clause 79. The method of clause 73, wherein prompting the user to take the blood sample includes periodic prompts for the user to take blood samples.

Clause 80. The method of clause 73, wherein the results indicate changes in health in response to the lifestyle change.

Clause 81. The method of clause 73, further comprising determining a channel as in any one of the preceding clauses and determining a change in the channel in response to the nutrition, an exercise regimen, or lifestyle change, and outputting the change in the channel to the user.

Clause 82. An apparatus comprising: a processor configured with instructions for: presenting at least one lifestyle change experiment to a user via a graphical user interface of a user device; receiving a selection of an experiment in a computing device; prompting, from the computing device and based on the selected experiment, a reminder to the user to perform a lifestyle change in accordance with the experiment; prompting, from the computing device, the user to take a blood sample; processing, in the computing device, spectroscopic data corresponding to the blood sample; and presenting results of the selected experiment based at least on the received spectroscopic data via the graphical user interface of the user device, wherein the results comprise a change in a value of a surrogate for a biomarker over a time interval.

Clause 83. The apparatus of clause 82, wherein the experiment comprises one or more of a metabolism experiment, a cardiovascular health experiment, an inflammation and immune function experiment, hematologic function experiment, a toxin experiment, a stress experiment, a saliva experiment or a fecal fat experiment.

Clause 84. A system, comprising: a spectrometer configured to perform a spectroscopy on a user's sample of blood by receiving the user's sample of blood contained within a sample holder, illuminating the user's sample of blood as the blood at least partially separates within the sample holder; and generating spectral data from the blood as the blood at least partially separates within the sample holder; and a network element communicatively coupled to the spectrometer and configured to process the spectral data to determine a surrogate for a biomarker, wherein the network element comprises a recommendation engine configured to generate a plurality of experiments for the user based on the surrogate.

Clause 85. The system of clause 84, wherein the experiments include consuming different edible substances to alter the surrogate in a subsequent spectroscopy on a subsequent sample of blood of the user.

Clause 86. The system of clause 84, wherein the recommendation engine is further configured to alert the user to perform one or more of the experiments.

Clause 87. The system of clause 84, wherein the recommendation engine is further configured to provide information pertaining to the plurality of experiments to the user.

Clause 88. The system of clause 84, wherein the recommendation engine is further configured to track progress of the user's experiments and changes in the user's surrogate.

Clause 89. The system of clause 84, wherein the network element is further configured to connect the user with other users to share results of the experiments.

Clause 90. The method, system, apparats or tangible medium of any one of the preceding clauses, wherein the surrogate is a level of fat in the blood and the biomarker is triglycerides.

Clause 91. The method, system, apparats or tangible medium of any one of the preceding clauses, wherein the channel comprises a spectral channel determined in response to a combination of spectral intensities of a plurality measured wavelength bands.

Clause 92. A processor configured with instructions to perform one or more steps of a method of any one of the preceding clauses.

Clause 93. A processor comprising the tangible medium of any one of the preceding clauses.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A method comprising:
receiving a blood sample;
generating a plurality of spectroscopic data points from the blood sample using a spectroscopy device, wherein the plurality of spectroscopic data points comprises spectrometer data of the blood sample taken over a time interval and the spectrometer data comprises intensities from a plurality of wavelength bins;
distributing the plurality of spectroscopic data points into a plurality of channels based on the plurality of wavelength bins, wherein each of the plurality of wavelength bins is associated with one or more of the plurality of channels and each of the plurality of channels comprises a combination of spectral measurement values from the plurality of wavelength bins;
analyzing the plurality of channels for each channel of the plurality of channels to determine a value for a surrogate for a biomarker as a function of time during the time interval;
determining if there is a significant change in the value of the surrogate over the time interval.

2. The method of claim 1, further comprising:
correlating the value of the surrogate over the time interval to the biomarker;
based on the correlation, determining whether to recommend a change to nutrition, exercise regimen, or lifestyle; and
if a change is recommended, generating a user interface on a device to present the change to a user of the device.

3. The method of claim 1, wherein the spectroscopy device comprises a near infrared spectroscopy device.

4. The method of claim 1, wherein the surrogate comprises a blood fat level and the biomarker comprises a triglyceride level.

5. The method of claim 1, further comprising separating the blood sample into blood cells and blood plasma prior to generating the plurality of spectroscopic data points.

6. The method of claim 5, further comprising determining the value of the fat level in the blood plasma twice in a period of a day and based on a difference between those two measurements, determining whether to recommend a change to nutrition, exercise regimen, or lifestyle.

7. The method of claim 6, wherein the difference between the two measurements is used as an indicator of one or more of nutrition or stress.

8. The method of claim 1, further comprising measuring a glucose level in the blood sample, and using the glucose level to determine if the change in the level of the surrogate should be used to determine whether to recommend a change to nutrition, exercise regimen, or lifestyle.

9. The method of claim 1, further comprising measuring a protein level in the blood sample, and using the protein level to determine if the change in the level of the surrogate should be used to determine whether to recommend a change to nutrition, exercise regimen, or lifestyle.

10. The method of claim 8, wherein if the glucose level is used to determine that the change in the level of the surrogate should not be used to determine whether to recommend a change to nutrition, exercise regimen, or lifestyle, then the method further comprises generating a notification on the device that the value of the surrogate should be remeasured at a later time.

11. The method of claim 9, wherein if the protein level is used to determine that the change in the level of the surrogate should not be used to determine whether to recommend a change to nutrition, exercise regimen, or lifestyle, then the method further comprises generating a notification on the device that the value of the surrogate should be remeasured at a later time.

12. The method of claim 1, further comprising receiving information describing a current nutrition, exercise regimen, or lifestyle, and wherein the recommended change is to a characteristic of the current nutrition, exercise regimen, or lifestyle.

13. The method of claim 1, wherein the surrogate is a sedimentation rate and the biomarker is a triglyceride level.

14. The method of claim 1, further comprising receiving information regarding the time of receiving the blood sample and a person's intake of food or exercising and the time of that intake or exercising; and
using the received information as training data for a model of the person's variation in the value of the surrogate over a period of time.

15. An apparatus comprising:
a processor configured with instructions for
receiving a blood sample;
generating a plurality of spectroscopic data points from the blood sample using a spectroscopy device, wherein the plurality of spectroscopic data points comprises spectrometer data of the blood sample taken over a time interval and the spectrometer data comprises intensities from a plurality of wavelength bins;
distributing the plurality of spectroscopic data points into a plurality of channels based on the plurality of wavelength bins, wherein each of the plurality of wavelength bins is associated with one or more of the plurality of channels and each of the plurality of channels comprises a combination of spectral measurement values from the plurality of wavelength bins;
analyzing the plurality of channels for each channel of the plurality of channels to determine a value for a surrogate for a biomarker as a function of time during the time interval.

16. The apparatus of claim 15, wherein the processor is configured for:
determining if there is a significant change in the value of the surrogate over the time interval;
correlating the value of the surrogate over the time interval to the biomarker;
based on the correlation, determining whether to recommend a change to nutrition, exercise regimen, or lifestyle; and
if a change is recommended, generating a user interface on a device to present the change to a user of the device.

17. The apparatus of claim 15, wherein the spectroscopy device comprises a near infrared spectroscopy device.

18. The apparatus of claim 15, wherein the surrogate comprises a blood fat level and the biomarker comprises a triglyceride level.

19. The apparatus of claim 15, further comprising separating the blood sample into blood cells and blood plasma prior to generating the plurality of spectroscopic data points.

20. The apparatus of claim 19, further comprising determining the value of the fat level in the blood plasma twice in a period of a day and based on a difference between those two measurements, determining whether to recommend a change to nutrition, exercise regimen, or lifestyle.
